SEQUENCE LISTING (4 sequences)

SEQ ID NO: 1 - DNA, 20 bp, Artificial Sequence
  Androgen Receptor forward primer
cggaagctgaagaaacttgg

SEQ ID NO: 2 - DNA, 20 bp, Artificial Sequence
  Adrogen Receptor reverse receptor
cgtgtccagcacacactaca

SEQ ID NO: 3 - DNA, 20 bp, Artificial Sequence
  PSA/KLK3 forward primer
tgagcctcctgaagaatcga

SEQ ID NO: 4 - DNA, 18 bp, Artificial Sequence
  PSA/KLK3 reverse primer
ttgcgcacacacgtcatt

(19) United States
(12) Patent Application Publication
(10) Pub. No.: US 2020/0276164 A1
(43) Pub. Date: Sep. 3, 2020

(54) PHARMACEUTICAL COMBINATIONS FOR THE TREATMENT OF CANCER

(71) Applicant: The Queen's University of Belfast, Belfast (GB)

(72) Inventors: David Waugh, BANGOR (GB); Daniel Longley, LISBURN (GB); Pamela Maxwell, NEWTOWNARDS (GB); Melissa LaBonte Wilson, PARKGATE (GB)

(73) Assignee: The Queen's University of Belfast, Belfast (GB)

(21) Appl. No.: 16/643,756

(22) PCT Filed: Aug. 31, 2018
(86) PCT No.: PCT/EP2018/073502

§ 371 Date: Mar. 2, 2020

(30) Foreign Application Priority Data
Aug. 31, 2017 (GB) 1713936.1

(51) Int. Cl. A61K 31/4166; C07K 16/22; C07K 16/24; A61P 35/00

(52) U.S. Cl. CPC A61K 31/4166; C07K 16/22; A61K 45/06; A61P 35/00; C07K 16/244

(57) ABSTRACT

This disclosure relates to an enhanced molecular-targeted approach that targets treatment-induced or native hypoxia present within cancers, specifically, but not limited to the treatment of metastatic prostate cancer with androgen deprivation therapy, such as anti-androgens. The disclosure describes the utility of combined inhibition of IL-8 and VEGF signalling to effect a combined therapeutic response of malignant disease, which is magnified under conditions of hypoxia. Thus, provided is a pharmaceutical combination for use in the treatment of cancer, the pharmaceutical combination comprising: a vascular endothelial growth factor (VEGF) signalling inhibitor; and an interleukin-8 (IL-8) signalling inhibitor; wherein said use comprises administration of the pharmaceutical combination to a patient receiving an anti-cancer therapy.

### TECHNICAL FIELD

This invention relates to an enhanced molecular-targeted approach that targets treatment-induced or native hypoxia present within cancers, specifically, but not limited to the treatment of metastatic prostate cancer with androgen deprivation therapy, such as anti-androgens. The invention describes the utility of combined inhibition of IL-8 and VEGF signalling to effect a combined therapeutic response of malignant disease, which is magnified under conditions of hypoxia.

### BACKGROUND ART

Inhibition of androgen signalling, through the use of anti-androgens or androgen receptor (AR)-targeted agents, remains the mainstay of therapeutic intervention in castrate-resistant prostate cancer (CRPC). The AR-targeted agent Enzalutamide (MDV3100) blocks androgen binding, inhibits AR nuclear translocation and prevents androgen-driven gene expression, yet provides only a modest overall survival benefit in “castrate-resistant” patients. The development of resistance has been attributed to the retention of active AR signalling, even in androgen-depleted conditions, as a result of amplification/over-expression of the AR gene, elevated levels of intra-prostatic androgen synthesis, or through the acquisition of mutations or expression of AR splice variants. However, the impact of the tumour microenvironment has rarely been considered in the context of castrate-resistance.

Anti-androgen therapy profoundly influences the microenvironment of the prostate gland. Loss of androgen signalling rapidly decreases prostatic blood flow and reduces microvessel density (MVD) of the prostate gland in rats. Rapid vascular atrophy and reduced MVD have also been observed in engrafted human prostate tumours following androgen withdrawal, caused-in-part by vascular endothelial cell apoptosis. Similarly, we previously demonstrated that administration of Bicalutamide, a clinically-approved AR antagonist induces a rapid and sustained hypoxia, followed by a secondary period of neo-angiogenesis in xenograft models.

Although treatment with AR-targeted agents effectively reduces proliferation of prostate cancer cells, treatment with Bicalutamide and Enzalutamide has been shown to increase their rate of invasion in vitro and potentiate the development of distant metastases in vivo. We have adopted the hypothesis that the development of treatment-associated hypoxia and its ensuing intracellular signalling may define a further mechanism of relapse to AR-targeted therapy and accelerate the outgrowth and dissemination of CRPC.

Therefore, the present invention has been developed with a view to providing an improved AR-targeted therapy by also targeting hypoxia-induced pro-angiogenic and/or pro-survival factors.

### SUMMARY OF THE INVENTION

Accordingly, in one aspect, the invention provides a pharmaceutical combination for use in the treatment of cancer, the pharmaceutical combination comprising:
a vascular endothelial growth factor (VEGF) signalling inhibitor; and
an interleukin-8 (IL-8) signalling inhibitor;
   wherein said use comprises administration of the pharmaceutical combination to a patient receiving an anti-cancer therapy.

The anticancer therapy may be therapy with a chemotherapeutic agent (chemotherapy) and/or radiotherapy. Optionally, the anticancer therapy is a chemotherapeutic agent and/or radiotherapy which cause one or more areas of hypoxia within a cancer treated by said chemotherapeutic agent and/or radiotherapy.

Optionally, the anticancer therapy is androgen deprivation therapy. Optionally, the androgen deprivation therapy causes one or more areas of hypoxia within a cancer treated by said androgen deprivation therapy. Optionally, the androgen deprivation therapy comprises treatment with an anti-androgen and/or an androgen signalling inhibitor. Preferably, the anti-androgen is an androgen receptor antagonist, such as enzalutamide (MDV3100).

Optionally, the present invention provides a pharmaceutical combination for use in the treatment of cancer, the pharmaceutical combination comprising:
a vascular endothelial growth factor (VEGF) signalling inhibitor; and
an interleukin-8 (IL-8) signalling inhibitor;
   wherein said use comprises administration of the pharmaceutical combination to a patient receiving a chemotherapeutic agent.

Optionally, the present invention provides a pharmaceutical combination for use in the treatment of cancer, the pharmaceutical combination comprising:
a vascular endothelial growth factor (VEGF) signalling inhibitor; and
an interleukin-8 (IL-8) signalling inhibitor;
   wherein said use comprises administration of the pharmaceutical combination to a patient receiving radiotherapy.

Optionally, the present invention provides a pharmaceutical combination for use in the treatment of cancer, the pharmaceutical combination comprising:
a vascular endothelial growth factor (VEGF) signalling inhibitor; and
an interleukin-8 (IL-8) signalling inhibitor;
   wherein said use comprises administration of the pharmaceutical combination to a patient receiving an androgen deprivation therapy, optionally an androgen receptor antagonist such as enzalutamide (MDV3100).

In another aspect, the present invention provides a pharmaceutical combination for use in the treatment of cancer, the pharmaceutical combination comprising:
an anti-cancer therapy;
a vascular endothelial growth factor (VEGF) signalling inhibitor; and
an interleukin-8 (IL-8) signalling inhibitor.

Optionally, the present invention provides a pharmaceutical combination for use in the treatment of cancer, the pharmaceutical combination comprising:
a chemotherapeutic agent;
a vascular endothelial growth factor (VEGF) signalling inhibitor; and
an interleukin-8 (IL-8) signalling inhibitor.

Optionally, the present invention provides a pharmaceutical combination for use in the treatment of cancer, the pharmaceutical combination comprising:
radiotherapy;
a vascular endothelial growth factor (VEGF) signalling inhibitor; and
an interleukin-8 (IL-8) signalling inhibitor.

Optionally, the present invention provides a pharmaceutical combination for use in the treatment of cancer, the pharmaceutical combination comprising:
an androgen deprivation therapy, optionally an androgen receptor antagonist such as enzalutamide (MDV3100);
a vascular endothelial growth factor (VEGF) signalling inhibitor; and
an interleukin-8 (IL-8) signalling inhibitor.

In another aspect, the present invention provides a method of treating cancer, comprising administering a pharmaceutical combination comprising:
a vascular endothelial growth factor (VEGF) inhibitor; and
an interleukin-8 (IL-8) inhibitor;
   to a patient receiving an anti-cancer therapy.

Optionally, the present invention provides a method of treating cancer, comprising administering a pharmaceutical combination comprising:
a vascular endothelial growth factor (VEGF) inhibitor; and
an interleukin-8 (IL-8) inhibitor;
   to a patient receiving a chemotherapeutic agent.

Optionally, the present invention provides a method of treating cancer, comprising administering a pharmaceutical combination comprising:
a vascular endothelial growth factor (VEGF) inhibitor; and
an interleukin-8 (IL-8) inhibitor;
   to a patient receiving radiotherapy.

Optionally, the present invention provides a method of treating cancer, comprising administering a pharmaceutical combination comprising:
a vascular endothelial growth factor (VEGF) inhibitor; and
an interleukin-8 (IL-8) inhibitor;
   to a patient receiving androgen deprivation therapy, optionally an androgen receptor antagonist such as enzalutamide (MDV3100).

In another aspect, the present invention provides a method of treating cancer, comprising administering a pharmaceutical combination comprising:
an anti-cancer therapy;
a vascular endothelial growth factor (VEGF) signalling inhibitor; and
an interleukin-8 (IL-8) signalling inhibitor;
   to a patient in need thereof.

Optionally, the present invention provides a method of treating cancer, comprising administering a pharmaceutical combination comprising:
a chemotherapeutic agent;
a vascular endothelial growth factor (VEGF) signalling inhibitor; and
an interleukin-8 (IL-8) signalling inhibitor;
   to a patient in need thereof.

Optionally, the present invention provides a pharmaceutical combination for use in the treatment of cancer, the pharmaceutical combination comprising:
radiotherapy;
a vascular endothelial growth factor (VEGF) signalling inhibitor; and
an interleukin-8 (IL-8) signalling inhibitor;
   to a patient in need thereof.

Optionally, the present invention provides a method of treating cancer, comprising administering a pharmaceutical combination comprising:
an androgen deprivation therapy, optionally an androgen receptor antagonist such as enzalutamide (MDV3100);
a vascular endothelial growth factor (VEGF) signalling inhibitor; and
an interleukin-8 (IL-8) signalling inhibitor;
   to a patient in need thereof.

In another aspect, the present invention provides a method of treating cancer in a patient receiving an anticancer therapy, optionally a chemotherapeutic agent, radiotherapy or an androgen deprivation therapy, the comprising:
inhibiting vascular endothelial growth factor (VEGF) signalling; and
inhibiting interleukin-8 (IL-8) signalling.

Optionally, the present invention provides a method of treating cancer, the comprising:
administering an anticancer therapy, optionally a chemotherapeutic agent, radiotherapy or an androgen deprivation therapy;
inhibiting vascular endothelial growth factor (VEGF) signalling; and
inhibiting interleukin-8 (IL-8) signalling,
   in a patient suffering from cancer.

Optionally, the present invention provides a method of treating cancer, the comprising:
antagonising an androgen receptor;
inhibiting vascular endothelial growth factor (VEGF) signalling; and
inhibiting interleukin-8 (IL-8) signalling.

Optionally, the method comprises administering an androgen receptor antagonist to antagonise the androgen receptor. Optionally, the method comprises administering a VEGF signalling inhibitor to inhibit the signalling effects of VEGF. Optionally, the method comprises administering an IL-8 signalling inhibitor to inhibit the signalling effects of IL-8.

In another aspect, the present invention provides a use of a pharmaceutical combination comprising:
a vascular endothelial growth factor (VEGF) inhibitor; and optionally
an interleukin-8 (IL-8) inhibitor;
   in the manufacture of a medicament for the treatment of cancer in a patient receiving an anti-cancer therapy.

Optionally, the present invention provides a use of a pharmaceutical combination comprising:
a vascular endothelial growth factor (VEGF) inhibitor; and optionally
an interleukin-8 (IL-8) inhibitor;
   in the manufacture of a medicament for the treatment of cancer in a patient receiving a chemotherapeutic agent.

Optionally, the present invention provides a use of a pharmaceutical combination comprising:
a vascular endothelial growth factor (VEGF) inhibitor; and optionally
an interleukin-8 (IL-8) inhibitor;
   in the manufacture of a medicament for the treatment of cancer in a patient receiving radiotherapy.

Optionally, the present invention provides a use of a pharmaceutical combination comprising:
a vascular endothelial growth factor (VEGF) inhibitor; and optionally
an interleukin-8 (IL-8) inhibitor;
   in the manufacture of a medicament for the treatment of cancer in a patient receiving an androgen deprivation therapy, optionally an androgen receptor antagonist such as enzalutamide (MDV3100).

In another aspect, the present invention provides a use of a pharmaceutical combination comprising:
an anti-cancer therapy;
a vascular endothelial growth factor (VEGF) inhibitor; and
an interleukin-8 (IL-8) inhibitor;
   in the manufacture of a medicament for the treatment of cancer.

Optionally, the present invention provides a use of a pharmaceutical combination comprising:
a chemotherapeutic agent;
a vascular endothelial growth factor (VEGF) inhibitor; and
an interleukin-8 (IL-8) inhibitor;
   in the manufacture of a medicament for the treatment of cancer.

Optionally, the present invention provides a use of a pharmaceutical combination comprising:
radiotherapy;
a vascular endothelial growth factor (VEGF) inhibitor; and
an interleukin-8 (IL-8) inhibitor;
   in the manufacture of a medicament for the treatment of cancer.

Optionally, the present invention provides a use of a pharmaceutical combination comprising:
an androgen deprivation therapy, optionally an androgen receptor antagonist such as enzalutamide (MDV3100);
a vascular endothelial growth factor (VEGF) inhibitor; and
an interleukin-8 (IL-8) inhibitor;
   in the manufacture of a medicament for the treatment of cancer.

In another aspect, the present invention provides a pharmaceutical combination for use in potentiating a therapeutic effect of an anti-cancer therapy in the treatment of cancer, the pharmaceutical combination comprising:
a vascular endothelial growth factor (VEGF) inhibitor; and
an interleukin-8 (IL-8) inhibitor.

Optionally, the present invention provides a pharmaceutical combination for use in potentiating a therapeutic effect of a chemotherapeutic agent in the treatment of cancer, the pharmaceutical combination comprising:
a vascular endothelial growth factor (VEGF) inhibitor; and
an interleukin-8 (IL-8) inhibitor.

Optionally, the present invention provides a pharmaceutical combination for use in potentiating a therapeutic effect of radiotherapy in the treatment of cancer, the pharmaceutical combination comprising:
a vascular endothelial growth factor (VEGF) inhibitor; and
an interleukin-8 (IL-8) inhibitor.

Optionally, the present invention provides a pharmaceutical combination for use in potentiating a therapeutic effect of an androgen deprivation therapy, optionally an androgen receptor antagonist such as enzalutamide (MDV3100), in the treatment of cancer, the pharmaceutical combination comprising:
a vascular endothelial growth factor (VEGF) inhibitor; and optionally
an interleukin-8 (IL-8) inhibitor.

In another aspect, the present invention provides a method for potentiating a therapeutic effect of an anti-cancer therapy in the treatment of cancer, comprising administering a pharmaceutical combination comprising:
a vascular endothelial growth factor (VEGF) inhibitor; and
an interleukin-8 (IL-8) inhibitor;
   to a patient receiving the anti-cancer therapy.

Optionally, the present invention provides a method for potentiating a therapeutic effect of a chemotherapeutic agent in the treatment of cancer, comprising administering a pharmaceutical combination comprising:
a vascular endothelial growth factor (VEGF) inhibitor; and optionally
an interleukin-8 (IL-8) inhibitor;
   to a patient receiving the chemotherapeutic agent.

Optionally, the present invention provides a method for potentiating a therapeutic effect of an androgen deprivation therapy, optionally an androgen receptor antagonist such as enzalutamide (MDV3100), in the treatment of cancer, comprising administering a pharmaceutical combination comprising:
a vascular endothelial growth factor (VEGF) inhibitor; and optionally
an interleukin-8 (IL-8) inhibitor;
   to a patient receiving the androgen deprivation therapy, optionally an androgen receptor antagonist such as enzalutamide (MDV3100).

Optionally, the cancer is a cancer characterised by one or more areas of hypoxia within the cancer, i.e. within the tumour mass. Without wishing to be bound by theory, it is understood that the hypoxic zones within the cancer may arise as a result of uncontrolled proliferation outstripping nutrient supply from the vasculature or may arise from disruption to the tumour vasculature following administration of a therapeutic agent or a vascular-disrupting drug. Optionally, the cancer is a cancer characterised by increased expression of VEGF and/or IL-8. Optionally, the cancer is selected from one or more of prostate cancer, breast cancer, colorectal cancer, pancreatic cancer, glioblastoma, lung cancer or gastric cancer. In particular, the cancer is selected from prostate cancer or breast cancer.

Optionally, the cancer is refractory, or substantially refractory, to treatment with a chemotherapeutic agent, radiotherapy and/or androgen deprivation therapy, such as androgen receptor antagonists and/or androgen signalling inhibitors. Optionally, the cancer is refractory, or substantially refractory, to treatment with enzalutamide (MDV3100).

Optionally, the prostate cancer is hormone-naïve, hormone-sensitive or castrate-resistant prostate cancer. Optionally, the prostate cancer is castrate-resistant prostate cancer, which cancer is known to be treated with androgen receptor antagonists and/or androgen signalling inhibitors. Alternatively, the prostate cancer is non-castrate prostate cancer. With regard to non-castrate prostate cancer, earlier treatment of this cancer with androgen signalling inhibitors has been undertaken in view of results from the STAMPEDE and LATITUDE Clinical Trials. Optionally, the prostate cancer is refractory, or substantially refractory, to treatment with a chemotherapeutic agent, radiotherapy and/or androgen deprivation therapy, such as androgen receptor antagonists and/or androgen signalling inhibitors. Optionally, the prostate cancer is refractory, or substantially refractory, to treatment with enzalutamide (MDV3100). Optionally, the present invention has utility in the treatment of localized prostate cancer, as well as in patients with no confirmed evidence of distant metastasis. Optionally, the pharmaceutical combination of the invention is administered to prostate cancer within primary site of the cancer or to an extra-prostatic site.

Optionally, the chemotherapeutic agent is selected from one or more of FOLFOX (folinic acid, fluorouracil and oxaliplatin) combination therapy, which chemotherapeutic agent is suitable for the treatment of metastatic colorectal cancer, Sunitinib or Lenalidomide, which chemotherapeutic agents have been used in the treatment of castrate-resistant prostrate cancer.

Optionally, the radiotherapy is selected from one or more of external beam radiation therapy, brachytherapy (sealed source radiotherapy), unsealed source radiotherapy (systemic radioisotope therapy), intraoperative radiotherapy, deep inspiration breath-hold radiotherapy or radionuclide therapy (e.g. radium-223).

Optionally, the androgen receptor antagonist is selected from one or more of enzalutamide (MDV3100), Apalutamide (ARN-509), Bicalutamide (Casodex), or Darulutamide.

Optionally, the androgen signalling inhibitor is selected from abiraterone-acetate, finasteride, dutasteride, leuprolide or gooserelin.

Optionally, the androgen receptor antagonist, such as enzalutamide, is to be administered at a pharmaceutically effective amount. Optionally, the androgen receptor antagonist, such as enzalutamide, is to be administered at a pharmaceutically effective amount of about 120-200 mg/day, optionally about 160 mg/day, via oral ingestion.

Optionally, radiotherapy is used in combination with the androgen deprivation therapy.

Administration of the androgen receptor antagonist may be by any suitable method known in the art, including subcutaneous, intramuscular, intradermal, intraperitoneal, intravenous, intranasal, or oral routes of administration. Preferably, the androgen receptor antagonist, such as enzalutamide, is for administration by the oral route of administration.

Optionally, the VEGF signalling inhibitor described herein comprises an antibody suitable for binding VEGF. Optionally, the antibody suitable for binding VEGF is a neutralising anti-VEGF antibody (VEGF nAb), exemplified by Avastin® (bevacizumab). It will be understood that a neutralising anti-VEGF antibody is an antibody that is capable of binding, optionally specifically binding, to a VEGF molecule and preventing or inhibiting the biological activity of the VEGF molecule. The preventing or inhibiting the biological activity of the VEGF molecule includes preventing or inhibiting the interaction of the VEGF molecule with its corresponding VEGF receptor, and/or preventing or inhibiting the signalling activity of the VEGF molecule such as through blockade of VEGR receptor (VEGFR1 and/or VEGFR2) activation. Activation of the receptor may be prevented by use of an antibody that binds, optionally specifically binds, to one or more epitopes on the receptor that prevent the binding of the natural (VEGF) ligand to its binding pocket on the receptor to promote activation.

Optionally, the VEGF signalling inhibitor described herein comprises an inhibitor or antagonist of the VEGF receptor (such as VEGFR1 and/or VEGFR2). Optionally, this may be an agent selected from one or more of Cediranib, Lenvantinib, Pazopanib, or Regorafenib. In the case of Pazopanib, this may be administered at a dosing range of about 100 mg/day to 1000 mg/day, optionally about 200 mg/day to 800 mg/day, through oral administration (e.g. tablet).

Optionally, the VEGF signalling inhibitor is to be administered at a pharmaceutically effective amount. In the case of Avastin® (bevacizumab), the VEGF signalling inhibitor may be administered at a pharmaceutically effective amount of about 10-20 mg/kg every 2-4 weeks, optionally about 15 mg/kg every 3 weeks, as an intravenous infusion.

Administration of alternate VEGF signalling inhibitors may be by any suitable method known in the art, including subcutaneous, intramuscular, intradermal, intraperitoneal, intravenous, intranasal, or oral routes of administration.

Optionally, the IL-8 signalling inhibitor described herein comprises an antibody suitable for binding IL-8. Optionally, the antibody suitable for binding IL-8 is a neutralising anti-IL-8 antibody (IL-8 nAb).

It will be understood that a neutralising anti-IL-8 antibody is an antibody that is capable of binding, optionally specifically binding, to an IL-8 molecule and preventing or inhibiting the biological activity of the IL-8 molecule. The preventing or inhibiting the biological activity of the IL-8 molecule includes preventing or inhibiting the interaction of the IL-8 molecule with its corresponding IL-8 receptor, and/or preventing or inhibiting the signalling activity of the IL-8 molecule such as through blockade of IL-8 receptor activation. Activation of the receptor may be prevented by use of an antibody that binds, optionally specifically binds, to one or more epitopes on the receptor that prevent the binding of the natural (IL-8) ligand to its binding pocket on the receptor to promote activation.

Optionally, the IL-8 signalling inhibitor described herein comprises an inhibitor or antagonist of the IL-8 receptor (such as CXCR1 and/or CXCR2), optionally a chemical species that acts as an inhibitor or antagonist of the IL-8 receptor, with unspecified selectivity to optionally block activation of the CXCR1 (IL8RA) and/or the CXCR2 (IL8RB) receptor. Several species have been developed including the use of agents like AZD5069 or SCH527123. Optionally, the inhibitor or antagonist of the IL-8 receptor comprises a selective and/or non-selective small molecule or peptide/peptidomimetic inhibitor of CXCR1 and/or CXCR2.

Optionally, the IL-8 signalling inhibitor is suitable to prevent or inhibit the activation of the CXCR1 (IL8RA) or CXCR2 (IL8RB) receptor. Optionally, the IL-8 signalling inhibitor comprises an antibody suitable for binding CXCR1 and/or CXCR2. It will be understood that a neutralising antibody to either CXCR1 or CXCR2 is an antibody that is capable of binding, optionally specifically binding, to the receptor protein and preventing or inhibiting the biological activity of the receptor. The preventing or inhibiting the biological activity of the specified receptors includes preventing or inhibiting the interaction of the IL-8 molecule or its associated ligands (CXCL1, CXCL2, CXCL5, CXCL6, CXCL8) with its corresponding receptor, and/or preventing or inhibiting the signalling activity of any of the associated ligands (CXCL1, CXCL2, CXCL5, CXCL6, CXCL8).

Optionally, the IL-8 signalling inhibitor is to be administered at a pharmaceutically effective amount and through acceptable routes of administration.

Administration of the IL-8 signalling inhibitor may be by any suitable method known in the art, including subcutaneous, intramuscular, intradermal, intraperitoneal, intravenous, intranasal, or oral routes of administration. Preferably, the IL-8 signalling inhibitor, such as a neutralising anti-IL-8 antibody, is for administration by the oral route of administration.

Optionally, the androgen receptor antagonist, VEGF signalling inhibitor, and IL-8 signalling inhibitor, of the pharmaceutical combination may be administered concurrently, consecutively, simultaneously, or at different times. Optionally, the androgen receptor antagonist, VEGF signalling inhibitor, and IL-8 signalling inhibitor, of the pharmaceutical combination may be administered together in a single pharmaceutical composition, or separately in separate pharmaceutical compositions.

In another aspect, the present invention provides a method of treating cancer, comprising administering a chemotherapeutic agent used in the treatment of the cancer, in combination with a vascular endothelial growth factor (VEGF) signalling inhibitor, and an interleukin-8 (IL-8) signalling inhibitor, to a patient suffering from cancer. Optionally, the treatment schedule may be realised by administration of an anti-IL-8 signalling inhibitor and an anti-VEGF signalling inhibitor, as described above, at the level to prevent inhibit signalling by the IL-8 and VEGF ligands, or sequester the IL-8 and VEGF ligands, or by preventing or inhibiting the activation of the corresponding receptors for the IL-8 and VEGF ligands.

In another aspect, the present invention provides a method of treating cancer, comprising administering radiotherapy in the treatment of the cancer, in combination with a vascular endothelial growth factor (VEGF) signalling inhibitor and an interleukin-8 (IL-8) signalling inhibitor, to a patient suffering from cancer. Optionally, the treatment schedule may be realised by administration of an anti-IL8 signalling inhibitor and an anti-VEGF signalling inhibitor, as described above, at the level to prevent inhibit signalling by the IL-8 and VEGF ligands, or sequester the IL-8 and VEGF ligands, or by preventing or inhibiting the activation of the specified receptors for the IL-8 and VEGF ligands.

In another aspect, the present invention provides a method of treating cancer, comprising administering androgen deprivation therapy in the treatment of the cancer, in combination with a vascular endothelial growth factor (VEGF) signalling inhibitor and an interleukin-8 (IL-8) signalling inhibitor, to a patient suffering from cancer. Optionally, the treatment schedule may be realised by administration of an anti-IL8 signalling inhibitor and an anti-VEGF signalling inhibitor, as described above, at the level to prevent inhibit signalling by the IL-8 and VEGF ligands, or sequester the IL-8 and VEGF ligands, or by preventing or inhibiting the activation of the specified receptors for the IL-8 and VEGF ligands.

In another aspect, the present invention provides a method of treating cancers that have zones of hypoxia, comprising treatment with a chemotherapeutic agent, radiotherapy or an androgen deprivation therapy, in combination with a vascular endothelial growth factor (VEGF) signalling inhibitor and an interleukin-8 (IL-8) signalling inhibitor, to a patient suffering from cancer. Optionally, the treatment schedule may be realised by administration of an anti-IL8 signalling inhibitor and an anti-VEGF signalling inhibitor, as described above, at the level to prevent inhibit signalling by the IL-8 and VEGF ligands, or sequester the IL-8 and VEGF ligands, or by preventing or inhibiting the activation of the specified receptors for the IL-8 and VEGF ligands.

The term “pharmaceutical combination” includes a combination of two or more therapeutic compositions or procedures suitable for the treatment of cancer. Thus, it will be understood, that administration of said therapeutic compositions to a patient, or carrying out said therapeutic procedures on a patient, may occur concurrently, consecutively, simultaneously, or at different times.

The term “antibody” includes a molecule from the subgroup of gamma globulin proteins which is also referred to as the immunoglobulins (Ig). Antibodies can, preferably, be of any subtype, i.e. IgA, IgD, IgE, IgM or, more preferably, IgG. Antibodies immobilised on particle carriers as described herein can be prepared by well-known methods using a purified polypeptide or a suitable fragment derived therefrom as an antigen. A fragment which is suitable as an antigen may be identified by antigenicity determining algorithms well known in the art. Such fragments may be obtained either by proteolytic digestion from target protein(s) or may be a synthetic peptide(s). Preferably, the antibody is a monoclonal antibody, a polyclonal antibody, a single chain antibody, a human or humanized antibody or primatized, chimerized or fragment thereof. Optionally, the antibody is a an antibody fragment, such as Fab, Fab′, (Fab′)2, Fv, scFv, Bis-scFv, minibody, Fab2, or Fab3 fragments, or a chemically modified derivative of any of these. An antibody of the present invention preferably binds specifically (i.e. does not cross react with other polypeptides or peptides) to a target protein(s) such as VEGF and/or IL-8. Specific binding can be tested by various well known techniques.

The terms “specific binding,” “specifically binds,” and similar may be understood to mean that an antibody exhibits appreciable affinity for a particular antigen or epitope and, generally, does not exhibit significant cross-reactivity with other antigens and epitopes. “Appreciable” or preferred binding includes binding with an affinity of at least (KD equal to or less than) 10⁻⁶ M, 10⁻⁷ M, 10⁻⁸ M, 10⁻⁹ M, 10⁻¹⁰ M, or 10⁻¹¹ M. An antibody that “does not exhibit significant crossreactivity” is one that will not appreciably bind to an undesirable entity (e.g., an undesirable proteinaceous entity). An antibody specific for a particular epitope will, for example, not significantly crossreact with other epitopes on the same protein or peptide. Specific binding can be determined according to any well known means for determining such binding. In some embodiments, specific binding is determined according to Scatchard analysis and/or competitive binding assays.

The term “inhibit” may be understood to mean a decrease of a biological activity. “Biological activity” includes inter- and intra-cellular signalling, transduction of a signal, etc. Inhibiting biological activity includes decreases the activity by 50%, 60%, 70%, 80%, 90%, 95% or 100% of the normal, uninhibited activity.

“Therapeutically effective amount” may be understood to mean a dose that produces the desired effect for which it is administered. The exact dose will depend on the purpose of the treatment, and will be ascertainable by one skilled in the art using known techniques (see, for example, Lloyd (1999) The Art, Science and Technology of Pharmaceutical Compounding). Efficacy can be measured in conventional ways, depending on the condition to be treated. For cancer therapy, efficacy can, for example, be measured by assessing the time to disease progression (TTP), or determining the response rates (RR). Therapeutically effective amount also refers to a target serum concentration, such as a trough serum concentration, that has been shown to be effective in suppressing disease symptoms when maintained for a period of time.

The phrase “treatment of cancer”, and the like, includes treatment of cancer to reduce tumour volume and/or reduce the rate of tumour growth. Tumour volume may be measured before or at the point of initial treatment and then again at any time after the treatment has begun, e.g. at day 28.

“Potentiate” may be understood to mean that administration of the VEGF inhibitor, and optionally IL-8 inhibitor, enhances or extends the therapeutic activity of the androgen receptor antagonist and/or results in a decreased amount of androgen receptor antagonist being required to produce a therapeutic effect. Thus, as will be understood, the therapeutically effective concentration of androgen receptor antagonist included in the pharmaceutical combinations of the present invention may be decreased as compared to an established effective, or ineffective, concentration for the androgen receptor antagonist when administered alone.

The term “patient” can include human and other mammalian subjects that receive the therapeutic treatment disclosed herein.

“Cancer” can include tumours, or neoplasms, which are benign, pre-malignant, or malignant. “Prostate cancer” can include carcinomas, including, carcinoma in situ, invasive carcinoma, metastatic carcinoma and pre-malignant conditions.

By “refractory”, and “substantially refractory”, it may understood that a cancer, optionally a prostate cancer, does not respond to (or is resistant to) treatment with an anti-cancer therapy, such as an androgen receptor antagonist and/or an androgen signalling inhibitor, or becomes unresponsive over time.

The androgen receptor (AR), also known as NR3C4 (nuclear receptor subfamily 3, group C, member 4), is a type of nuclear receptor that is activated by binding either of the androgenic hormones, testosterone or dihydrotestosterone, in the cytoplasm and then translocating into the nucleus. AR expression is maintained throughout prostate cancer progression, and the majority of androgen-independent or hormone refractory prostate cancers express AR.

The androgen receptor antagonist, a VEGF signalling inhibitor, and an IL-8 signalling inhibitor which may be comprised in the pharmaceutical combination disclosed herein may further comprise a pharmaceutically acceptable carrier. The term “pharmaceutically acceptable” means approved by a regulatory agency of, for example, the USA or EU. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Pharmaceutical compositions comprising the androgen receptor antagonist, a VEGF signalling inhibitor, and/or an IL-8 signalling inhibitor can, if desired, also contain minor amounts of wetting or emulsifying agents, or pH buffering agents. These compositions may take the form of solutions, suspensions, emulsion, tablets, pills, capsules, powders, sustained-release formulations and the like. The composition may be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. The composition may be formulated in accordance with routine procedures as a pharmaceutical composition adapted for intravenous, subcutaneous, or intramuscular administration to human beings. Where necessary, the composition may also include a solubilizing agent and a local anesthetic such as lidocaine to ease pain at the site of the injection. If a composition is to be administered by infusion, it may be dispensed with an infusion bottle containing sterile pharmaceutical grade water or saline. If a composition is administered by injection, an ampoule of sterile water for injection or saline can be provided so that the ingredients may be mixed prior to administration.

Actual dosage levels of the androgen receptor antagonist, VEGF signalling inhibitor, and IL-8 signalling inhibitor in the pharmaceutical combinations provided herein may be varied so as to obtain an amount of each of these components which is effective to achieve the desired therapeutic response for a particular patient, composition, and mode of administration, without being toxic to the patient. The selected dosage level will depend upon a variety of pharmacokinetic factors including the activity of the particular compositions employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the composition required. For example, the physician or veterinarian could start doses of the androgen receptor antagonist, VEGF, inhibitor, and IL-8 signalling inhibitor at levels lower than that required to achieve the desired therapeutic effect and gradually increasing the dosage until the desired effect is achieved. In general, a suitable daily dose of compositions provided herein will be that amount of the androgen receptor antagonist, VEGF signalling inhibitor, and IL-8 signalling inhibitor which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of a therapeutic composition may be administered as two, three, four, five, six or more sub-doses administered separately at appropriate intervals throughout the day.

By “about”, as used herein, it is meant that may be ±20% of the recited value, optionally±10% of the recited value, optionally±5% of the recited value, further optionally the value may be precisely the recited value.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure and so forth. Similarly, a reference to “an inhibitor” includes one or more inhibitors.

### BRIEF DESCRIPTION OF THE DRAWINGS

The embodiments of the present invention will now be described, by way of example, with reference to the accompanying drawings, in which:

FIG. 1 illustrates that MDV3100 treatment directly affects tumour vasculature. (A) Graph illustrating measured intra-tumoural oxygenation levels in growing MDV3100-treated LNCaP tumours, in the absence and presence of anti-IL-8 nAb (50 μg/ml) and/or anti-VEGF nAb (100 μg/ml). Values shown are mean (±SD). (B) Bar graph presenting time-dependent changes in tumour vessel density over time in MDV3100-treated LNCaP tumours, in the absence or presence of anti-IL-8 nAb (50 μg/ml) and/or anti-VEGF nAb (100 μg/ml). Stereological methods were used to analyze change in vessel density over time in all treatment groups and values were used to calculate percentage area covered by vessels. (C) Top Panel Bar graph presenting qPCR data demonstrating detectable AR mRNA expression by LNCaP and HUVEC cells. Data shown are the mean (±SEM) of four individual experiments. Bottom Panel Representative images of prostate tumour stained with (A) androgen receptor (B) haematoxylin and eosin (20× magnification). Endothelial cells and vessels are marked by black arrows in the prostate tumour. (D) Top Panel Bar graph illustrating the effect of MDV3100 on viability of HUVEC cells over 72 h. Control cells were treated with an equivalent volume of DMSO. Viability was determined by MTT assay. Data shown are the mean (±SEM) of three individual experiments. Bottom Panel Graph demonstrating the percentage of HUVEC cells undergoing apoptosis following 72 h treatment with MDV3100 (10 μM). Control cells were treated with an equivalent volume of DMSO. Apoptosis was assessed by Annexin V/PI staining. (E) Top Panel Representative images and bar graph demonstrating the effect of MDV3100 (10 μM) on angiogenesis over a 10 day period. Control cells were treated with an equivalent volume of DMSO. The number of junctions was measured using AngioSys 2.0 software. Data presented are the mean (±SEM) of 8 fields of view. Bottom Panel Bar graphs demonstrating the effect of MDV3100 (10 μM) on colonization of PC3 cells over five days in the CAM assay. Control cells were treated with an equivalent volume of DMSO. Data shown are the mean (±SEM) of 17 embryos for DMSO and 23 embryos for the MDV3100. For all data, statistically significant differences were determined using a Student's two-tailed t-test or Mann-Whitney U test:* p<0.05, ** p<0.01, *** p<0.001.

FIG. 2 illustrates that hypoxia differentially induces AR expression and activation in PTEN-deficient and PTEN-expressing prostate cancer cells. (A) Bar graph presenting the results of qPCR analysis, demonstrating the effect of hypoxia on AR expression in androgen dependent prostate cancer cells. Data shown are the mean (±SEM) of six individual experiments. (B) Immunoblots demonstrating the effect of hypoxia on expression of the AR in LNCaP cells (top), and AR and AR-V7 expression in 22Rv1 cells (bottom). Blots shown are representative of 3 individual experiments. Equal loading was assessed using GAPDH. (C) Left Panel Bar graph presenting luciferase reporter assays, demonstrating the time-dependent effect of hypoxia on AR transcriptional activity in LNCaP and 22Rv1 cells. Data shown are the mean (±SEM) of five independent experiments. Right Panel Bar graph illustrating the comparative levels of AR-driven luciferase activity detected in LNCaP and 22Rv1 cells at baseline and following exposure to hypoxia (6 h). Data shown are the mean (±SEM) of three independent experiments. (D) Bar graph presenting qPCR analysis demonstrating up-regulation of PSA expression in LNCaP, but not 22Rv1, cells in response to hypoxia. Data shown are the mean (±SEM) of four independent experiments. (E) Immunocytochemistry analysis of AR distribution in LNCaP cells (top) and 22Rv1 cells cultured under normal or hypoxic conditions (6 h). Images present a merged image, DAPI staining, and AR-related fluorescence. Scale bar=20 μm. (F) Bar graph illustrating the effect of MDV3100 (10 μM) on viability of LNCaP cells. Control cells were treated with an equivalent volume of DMSO. Cells were treated under normoxic or hypoxic conditions for 72 h. Data shown are the mean (±SEM) of three independent experiments. (G) Immunoblot demonstrating the effect of MDV3100 (10 μM) on PARP expression under normoxic and hypoxic condition. Control cells were treated with an equivalent volume of DMSO. Cells were treated for 72 h. Blots shown are representative of 3 individual experiments. Equal loading was assessed using GAPDH. For all experiments statistical analysis was carried out using a Student's two-tailed t-test test or Mann-Whitney U test: * p<0.05, ** p<0.01, *** p<0.001.

FIG. 3 illustrates that hypoxia-induced signalling sustains disease-progressing signalling pathways in androgen dependent prostate cancer cells. (A) Bar graphs presenting qPCR data demonstrating the effect of HIF-1α, NF-κB (RelA) or combined HIF-1α/RelA-siRNA transfections on the hypoxia-induced expression of VEGF (top panel), CAIX (middle panel), and BCL2 (bottom panel) in LNCaP cells. Control cells were transfected with equal concentrations of non-targeting oligonucleotide sequences. Data shown are the mean (±SEM) of four individual experiments. (B) Bar graphs presenting qPCR data demonstrating sustained upregulation of VEGF (Top Panel) and IL-8 (Bottom Panel) expression following exposure of hypoxic LNCaP cells to MDV3100 (10 μM). Control cells were treated with an equivalent volume of DMSO. Data shown are the mean (±SEM) of three individual experiments. (C) Bar graphs presenting ELISA data demonstrating that MDV3100 (10 μM) does not reverse the effects of hypoxia on secretion of VEGF (Top Panel) and IL-8 (Bottom Panel). Control cells were treated with an equivalent volume of DMSO. Data shown are the mean (±SEM) of at least three individual experiments. (D) Bar graphs presenting qPCR data demonstrating sustained upregulation of Bcl-2 expression following exposure of hypoxic LNCaP cells to MDV3100 (10 μM). Control cells were treated with an equivalent volume of DMSO. Data shown are the mean (±SEM) of three individual experiments. (E) Bar graphs presenting qPCR data demonstrating sustained upregulation of CAIX expression following exposure of hypoxic LNCaP cells to MDV3100 (10 μM). Control cells were treated with an equivalent volume of DMSO. Data shown are the mean (±SEM) of three individual experiments. For all experiments statistical analysis was carried out using a Student's two-tailed t-test test or Mann-Whitney U test: * p<0.05, ** p<0.01, NS, not significant.

FIG. 4 illustrates that inhibition of IL-8 and VEGF attenuates stress-induced signalling in hypoxic PTEN-deficient LNCaP cells. For all data, cells were treated with anti-IL-8 nAb at a concentration of 5 μg/ml and/or anti-VEGF nAb at 10 μg/ml. Control cells were treated with the highest concentration of isotype-matched human IgG antibody. (A) Bar graph presenting qPCR data demonstrating the effect of administering anti-IL-8 nAb and/or anti-VEGF nab on the hypoxia (6 h)-induced expression of VEGF, IL-8 (Top Panel), AR and PSA/KLK3 (Bottom Panel) mRNA in LNCaP cells. Data shown are the mean (±SEM) of at least three individual experiments. (B) Bar graph presenting ELISA data demonstrating the effect of administering anti-IL-8 nAb and/or anti-VEGF nAb on the hypoxia-induced expression of VEGF (Top Panel) or IL-8 (Bottom Panel) mRNA in LNCaP cells. Data shown are the mean (±SEM) of four individual experiments. (C) Top Panel Immunoblot demonstrating the effect of anti-IL-8 antibody and/or anti-VEGF antibody on the hypoxia-induced expression of AR and AR-V7 in hypoxic 22Rv1 cells. Blots shown are representative of three individual experiments. Equal loading was assessed using GAPDH. Bottom Panel Bar graph presenting qPCR data demonstrating the effect of anti-IL-8 nAb and/or anti-VEGF nAb on the hypoxia-induced expression of PSA/KLK3 in 22Rv1 cells. Cells were treated with nAbs in the presence of hypoxia for 6 h. Data presented are the mean (±SEM) of four independent experiments. (D) Top Panel Bar graph presenting qPCR data demonstrating the effect of anti-IL-8 nAb and/or anti-VEGF nAb on the hypoxia-induced expression of BCL2 mRNA in LNCaP cells. Data presented are the mean (±SEM) of three independent experiments. Bottom Panel Bar graph demonstrating the effect of anti-IL-8 nAb and/or anti-VEGF nAb on viability of hypoxic LNCaP and 22Rv1 cells over 72 h. Data shown are the mean (±SEM) of four independent experiments. For all experiments statistical analysis was carried out using a Student's two-tailed t-test test or Mann-Whitney U test: * p<0.05, ** p<0.01.

FIG. 5 illustrates that inhibition of VEGF and IL-8 signalling attenuates angiogenesis under conditions of treatment-induced hypoxia and enhances the response to MDV3100. (A) Representative images and bar graph demonstrating the effect of VEGF (2 ng/ml) or rh-IL-8 (3 nM) on angiogenesis over 10 day periods. Suramin (20 μM) was included as a negative control. (B) Representative images and bar graph demonstrating the effect conditioned media (CM) harvested from LNCaP cells cultured in hypoxia for 24 h, in the presence or absence of anti-IL-8 nAb (5 μg/ml) and/or anti-VEGF nAb (10 μg/ml) on angiogenesis over a 10 day period. For both experiments, the number of junctions was measured using AngioSys 2.0 software. Data presented are the mean (±SEM) of 8 fields of view. (C) Graph presenting tumour growth data, obtained by measuring tumour volume every 2 days over a period of 28 days. Male Balb/c SCID mice bearing tumours of 150-200 mm² were assigned to the following treatment groups: vehicle-only, MDV3100 (4 mg/kg), MDV3100 (4 mg/kg)+IgG control (150 μg/ml), MDV3100 (4 mg/kg)+anti-VEGF nAb (100 μg/ml) and MDV3100 (4 mg/ml)+anti-VEGF (100 μg/ml) and anti-IL-8 (50 μg/ml) nAbs. MDV3100 was given each day and neutralizing antibodies were administered 3×/week via i.p. injection for the duration of the study. The data points represent the mean (±SEM). (D) Bar graph representing the average tumour weight (mg) at completion of the study. Values shown are mean (±SEM) of at least three animals per treatment group. (E) Kaplan-Meier analysis of survival was constructed using the end-point definition as “time taken for tumor volume to reach twice that when treatment commenced”. For all experiments statistical analysis was carried out using a Student's two-tailed t-test test, Mann-Whitney U test or 2-way ANOVA with Bonferroni post-tests: * p<0.05, ** p<0.01, *** p<0.001.

FIG. 6 illustrates that VEGF and IL-8 plays a role in resistance to MDV3100. (A) Results of RNA-sequencing analysis of MDV3100-sensitive versus MDV3100-resistant, LNCaP-derived models. Data shown illustrates the levels of VEGF (left panel) and IL-8 (right panel) in MDV3100-sensitive line V16 versus two MDV3100-resistant lines, MR49F and MR49C. (B) Left Panel Bar graphs presenting qPCR data demonstrating increased AR mRNA expression in LNCaP-EnzR cells relative to LNCaP-Par cells. Data shown are the mean (±SEM) of three independent experiments. Right Panel Immunoblot demonstrating increased AR expression by LNCaP-EnzR cells relative to LNCaP-Par cells. Equal loading was assessed using GAPDH. Blots shown are representative of three individual experiments. (C) Bar graphs presenting qPCR data demonstrating increased expression of VEGF (Left Panel) and IL-8 (Right Panel) mRNA in LNCaP-EnzR cells relative to LNCaP-Par cells. Data shown are the mean (±SEM) of at least three independent experiments. (D) Bar graphs presenting ELISA data demonstrating increased secretion of VEGF (Left Panel) and IL-8 (Right Panel) in LNCaP-EnzR cells relative to LNCaP-Par cells. Data shown are the mean (±SEM) of at least four individual experiments. (E) Bar graphs demonstrating the effect of combined treatment with anti-IL-8 nab (5 μg/ml) and anti-VEGF nAb (10 μg/ml) on the response of LNCaP-Par and LNCaP-EnzR cells to MDV3100 (10 μM) under normoxic (top panel) or hypoxic (bottom panel) conditions. Data presented is the mean (±SEM) of 4 independent experiments. (F) Immunoblots demonstrating the effect of combined treatment with anti-IL-8 nAb (5 μg/ml) and anti-VEGF nAb (10 μg/ml) on expression of PARP, AR, c-FLIP and Bcl-2 following treatment of LNCaP-EnzR cells with MDV3100 (10 μM) for 72 h. Equal loading was assessed using GAPDH. Blots shown are representative of three independent experiments. F or all experiments statistical analysis was carried out using a Student's two-tailed t-test test or Mann-Whitney U test: * p<0.05, ** p<0.01, *** p<0.001.

FIG. 7 illustrates that AR-independent PC3 prostate cancer cells do not respond to MDV3100. (A) Graph illustrating the effect of increasing concentrations of MDV3100 upon the viability of PC3 cells over 72 h. Control cells were treated with an equivalent amount of DMSO vehicle. Data presented are the mean (±SEM) of three independent experiments. (B) Bar graphs presenting ELISA data demonstrating the effect of MDV3100 (10 μM, 24 h) on secretion of VEGF (left panel) and IL-8 (right panel). Control cells were treated with an equivalent volume of DMSO. (C) Representative images and bar graph demonstrating the effect conditioned media (CM) harvested from PC3 cells, cultured in the presence or absence MDV3100 (10 μM), on angiogenesis over a 10 day period. For both experiments, the number of junctions was measured using AngioSys 2.0 software. Data presented are the mean (±SEM) of 8 fields of view.

FIG. 8 illustrates that targeting HIF-1 and NF-κB signalling attenuates expression of AR in LNCaP cells. (A) Immunoblot validating down regulation of the RelA(p65) protein expression using 10 nM, nM and 50 nM for 72 h in LNCaP cells. RelA was used at 50 nM in further experiments. (B) Bar graphs presenting the results of qPCR analysis demonstrating the effect of HIF-1α siRNA, NF-κB siRNA or HIF-1α/NF-κB siRNA on the hypoxia-induced expression of AR (top panel) and PSA/KLK3 (bottom panel) mRNA in LNCaP cells. Control cells were treated with the highest concentration of non-targeting oligonucleotide sequences. Data shown are the mean (±SEM) of four individual experiments. Data shown are the mean (±SEM) of four individual experiments. (C) Immunoblot demonstrating the effect of HIF-1 siRNA, NF-κB siRNA or HIF-1-/NF-κB-siRNA on the hypoxia-induced expression of AR protein levels LNCaP cells. Control cells were treated with the highest concentration of non-targeting oligonucleotide sequences. Statistical analysis was carried out using a Student's two-tailed t-test or Mann-Whitney U test: * p<0.05.

FIG. 9 illustrates that the response of C4-2B cells to MDV3100 is attenuated in the presence of hypoxia. (A) Graph illustrating the effect of increasing concentrations of MDV3100 for 72 h upon the viability of C4-2B cells. Control cells were treated with an equivalent amount of DMSO vehicle. Data presented are the mean (±SEM) of four independent experiments. (B) Bar graph illustrating the relative sensitivity of C4-2B cells to MDV3100 (10 μM, 72 h) compared to LNCaP cells. Control cells were treated with an equivalent amount of DMSO vehicle. Data presented are the mean (±SEM) of four independent experiments. (C) Bar graph demonstrating the response of C4-2B cells to MDV3100 (10 μM) under hypoxic conditions for 72 h. Control cells were treated with an equivalent amount of DMSO vehicle. Data presented are the mean (±SEM) of three independent experiments. Statistical significance was asses using a Student's two-tailed t-test: **, p<0.01; ***, p<0.001.

FIG. 10 illustrates that IL-8 and VEGF neutralizing antibodies do not attenuate in vitro vessel formation. Representative images and bar graph demonstrating the effect of anti-IL-8 nAb (5 μg/ml) and/or anti-VEGF nAb (10 μg/ml) on angiogenesis over 10 days. The number of junctions was measured using AngioSys 2.0 software. Data presented are the mean (±SEM) of 8 fields of view. Statistical significance was asses using a Student's two-tailed t-test: ***, p<0.001.

FIG. 11 illustrates that LNCaP-EnzR cells are resistant to MDV3100 and this can be partially reversed by targeting VEGF and IL-8 signalling. (A) Left panel Bar graph illustrating the effect of MDV3100 (10 μM) on viability of LNCaP-Par and LNCaP-EnzR cells over 72 h. Viability was determined by MTT assay. Data shown are the mean (±SEM) of three individual experiments. Right Panel Bar graphs presenting cell cycle analysis, determined by PI staining of MDV3100-treated (10 μM) LNCaP-Par and LNCaP-EnzR cells. Control cells were treated with an equivalent volume of DMSO. Data shown are the mean (±SEM) of four independent experiments. (B) Immunoblots illustrating expression of PARP, AR, c-FLIP and Bcl-2 following exposure of LNCaP-EnzR cells to MDV3100 (10 μM) for 72 h. Blots shown are representative of three independent experiments. Loading was assessed using GAPDH. (C) Bar graphs demonstrating the effect of co-treatment of LNCaP-Par and LNCaP-EnzR cells with MDV3100 (10 μM) in combination with anti-IL-8 nAb (5 μg/ml) and anti-VEGF nAb (10 μg/ml) on cell cycle. Control cells were treated with an equivalent volume of DMSO. Data shown are the mean (±SEM) of four independent experiments. Statistical significance was asses using a Student's two-tailed t-test: **, p<0.01.

### DETAILED DESCRIPTION

### Materials and Methods

### Cell Culture

Authenticated PC3 (ATCC CRL-1435), LNCaP (ATCC CRL-1740) and 22Rv1 (ATCC CRL-2505) cells were cultured as described in Seaton et al. (Carcinogenesis 2008; 29: 1148-56). C4-2B cells were maintained in RPMI 1640 supplemented with 10% fetal calf serum (FCS). MDV3100-sensitive (LNCaP-Par) and resistant (LNCaP-EnzR) cells were obtained from Prof. Vander Griend, University of Chicago, Chicago, Ill., and were cultured as described in Kregel et al. (Oncotarget 2016; 7: 26259-74). HUVEC (ATCC CRL-1730) cells were maintained in Endothelial Cell Growth Medium (#CC-3162, Lonza, UK). For experiments involving hypoxia (0.1% 02), cells were cultured as previously reported (Maxwell et al., Oncogene 2007; 26: 7333-45). Human IL-8 monoclonal antibody (#MAB208), human VEGF mAb (#AB293NA) and isotype-matched IgG (#MAB002) were obtained from R&D Systems (UK). MDV3100 (#S1250) was obtained from Selleckchem (Germany). Cells were regularly tested to ensure they were free of mycoplasma contamination.

### Cell Viability Assays

Cell viability was assessed by 3-(4,5-dimethylthiazol-2-yl)-2,5-diphenyltetrazolium bromide (MTT) assay as previously described (Maxwell et al., Oncogene 2007; 26: 7333-45).

### Luciferase Assays

AR activity was measured by luciferase reporter assay, as previously described (Seaton et al., Carcinogenesis 2008; 29: 1148-56).

siRNA Transfection

HIF1α (#M-004018-05) or RelA (#M-003533-02) were targeted using oligonucleotide pools (GE Dharmacon, CO, USA) as previously described (Maxwell et al., Oncogene 2007; 26: 7333-45). Non-targeting-(NT) transfections were at the same concentrations as the siRNAs.

### Real-Time PCR

Nucleic acid samples were prepared and analyzed as previously described (Maxwell et al., Oncogene 2007; 26: 7333-45; Maxwell et al., Eur Urol 2013; 64: 177-88). Primer sequences were as follows: AR: Forward, 5′-CGGAAGCTGAAGAAACTTGG-3′ (SEQ ID NO: 1); Reverse, 5′-CGTGTCCAGCACACACTACA-3′ (SEQ ID NO: 2); PSA/KLK3: Forward, 5′-TGAGCCTCCTGAAGAATCGA-3′ (SEQ ID NO: 3); Reverse, 5′-TTGCGCACACACGTCATT-3′ (SEQ ID NO: 4). Primer sequences for 18s, BCL2, CAIX, IL-8, and VEGF have been previously reported (Maxwell et al., Oncogene 2007; 26: 7333-45; Lekas et al., Urol Res 1997; 25: 309-14).

### Immunoblotting

Immunoblotting was performed as previously described (McCourt et al., Clin Cancer Res 2012; 18:3822-33). Antibodies were obtained as follows: AR, Millipore (#PG-21, UK); AR-V7 (#ab198394) & PARP (#14-6667-82), Abcam (UK); Bcl-2 (#4223), Cell Signalling Technology (The Netherlands); c-FLIP (#AG-20B-0056), Adipogen (Switzerland); RELA(p65) (#sc-372), Santa Cruz (CA, USA); GAPDH (#MCA4740), Biorad (UK).

### ELISA

Secreted IL-8 (#M1918, Pelikine, Mast Group, UK), and VEGF (#DY293B, R&D Systems, UK) were measured by ELISA as per manufacturer's instructions.

### Androgen Receptor Immunofluorescence

Cells were seeded on coverslips, cultured under normoxic or hypoxic conditions, fixed, permeabilised, and blocked overnight. AR was visualized using primary anti-AR antibody (#PG-21, Millipore, UK) and Alexa fluor-568 secondary antibody (#A11036, Molecular Probes, UK). ProLong Gold Antifade Reagent with DAPI (#P36941, Thermo Scientific, UK) was used to visualize the nuclei. Cells were viewed under a Nikon Eclipse Ti—S fluorescent microscope and images captured using NIS-Elements software.

### RNA-Seq

RNA-Seq analysis was performed as previously described (Yamamoto et al., Clin Cancer Res 2015; 21: 1675-87).

### Cell Cycle Analysis

Cell cycle analysis was carried out by PI staining as previously described (Wilson et al., J Pharmacol Exp Ther 2008; 327: 746-59).

### IHC

Immunohistochemistry was performed for AR (ab9474; Abcam, UK) and CD31 (ab28364; Abcam, UK) on 4 μm tissue microarray sections of formalin-fixed paraffin-embedded prostate cancer specimens. Standard processing steps for each antibody were in accordance with manufacturer's instructions. Briefly, heat-induced antigen retrieval with epitope retrieval ER1 solution (#AR9961, Leica Biosystems, UK) was performed prior to incubation with primary antibody. Slides were washed with Bond washing buffer (#AR9590, Leica Biosystems, UK) and incubated with secondary antibody (Bond Polymer Refine kit #DS9800; Leica Biosystems, UK). Subsequently chromogenic detection was achieved by incubation with 3,30-diaminobenzidine (DAB) followed by Bond DAB enhancer (#AR9432, Leica Biosystems, UK). All slides were counterstained with haematoxylin and dehydrated through ethanol to xylene before mounting.

### In Vitro Angiogenesis Assay

The V2A angiogenesis assay (#ZHA-4000, Cellworks, UK) was carried out according to the manufacturer's instruction. Cells were allowed to settle for 4 days prior to treatment. Treatments were carried out in duplicate and were replenished every 2 days for 10 days. For treatments involving conditioned media, PC3 or LNCaP cells were treated as required for 24 h, media harvested and stored at −20° C. Vessels were visualized at 4× magnification. Vessel density was measured in four fields per well using AngioSys 2.0 software (Cellworks, UK).

### Tumour Cell Extravasation and Colonization in the Chick Embryo Experimental Metastasis Model

Experimental metastasis assays were performed in chick embryos as described (Deryugina et al., Histochem Cell Biol 2008; 130: 1119-30). On day 12 of incubation, PC3 cells were injected directly into the blood circulation through an allantoic vein (1×10⁵ cells in 0.1 mL serum-free medium per embryo). MDV3100 was delivered i.v. on days 1 and 2 after cell inoculations (0.1 mL of a 20 μM solution per embryo). On day 5, portions of the CAM were harvested to quantify the number of human tumour cells by human-specific Alu-qPCR as previously described (Deryugina et al., Cancer Res 2005; 65: 10959-69), using a standard curve generated by serial dilutions of human tumour cells within a constant number (1×10⁶) of chick embryo fibroblasts.

### In Vivo Models

In vivo experiments were conducted in accordance with the Animal (Scientific Procedures) Act 1986 and the UKCCCR Guidelines (2010) for the welfare of animals in experimental neoplasia.

### Drug Administration

MDV3100 (in 0.1% DMSO in corn oil) was administered orally (p.o). Vehicle control (VC) or MDV3100 (4 mg/kg, equivalent to 100 mg/day in men) was administered daily. Human IL-8 and VEGF nAbs were administered 3×/week via intraperitoneal (i.p.) injection, at a final concentration of 50 μg/ml and 100 μg/ml, respectively. IgG control was at a final concentration of 150 μg/ml.

### Xenograft Study

LNCaP cells (1×10⁷ in Matrigel) were implanted on the rear dorsum of 8-10-week-old male Balb/c SCID mice. When tumour volume was 150-200 mm³, mice were randomly assigned to treatment groups (3-5 per group). Tumour dimensions were measured as previously described (9). Mice were sacrificed at day 28 or day 35. Tumour growth over 28 days (% T/C) was calculated using the equation: % T/C=(mean tumour volume on day 28−mean starting volume)/(mean control tumour volume on day 28−mean control starting volume)×100.

### Dorsal Skin Flap Model

A viewing chamber was attached to a raised skin flap on the dorsal surface of the mouse (Balb/c SCID) and a fragment (˜0.5 mm) of LNCaP tumour was placed on the microvascular as previously described (9). Tumour vasculature was imaged weekly/4 weeks using a stereomicroscope. Image analysis was carried out using Touptek software (Touptek Photonics, China).

### Tumour Oxygenation Measurement

Tumour oxygenation was measured once weekly as previously described (Ming et al., Int J Cancer 2013; 132:1323-32).

### Statistical Analysis

Data was analyzed using GraphPad Prism software. Statistical significance between groups was determined using a two-tailed Student's t-test, Mann Whitney U-test or 2-way ANOVA with Bonferroni post-tests, as appropriate.

### Results

### MDV3100 Treatment Promotes Temporal Hypoxia and Delays Angiogenesis In Vivo Through Endothelial Cell Catastrophe

Anti-androgen therapy is associated with reductions in MVD and tumour oxygen levels while expression of the pro-angiogenic factors IL-8 and VEGF-A (VEGF) is altered in response to Bicalutamide-promoted hypoxia in vivo. The intent of this study was to examine the importance of these treatment-associated, hypoxia-inducible factors in modulating the oxygen tension and the vascularity of the microenvironment following Enzalutamide (MDV3100) therapy.

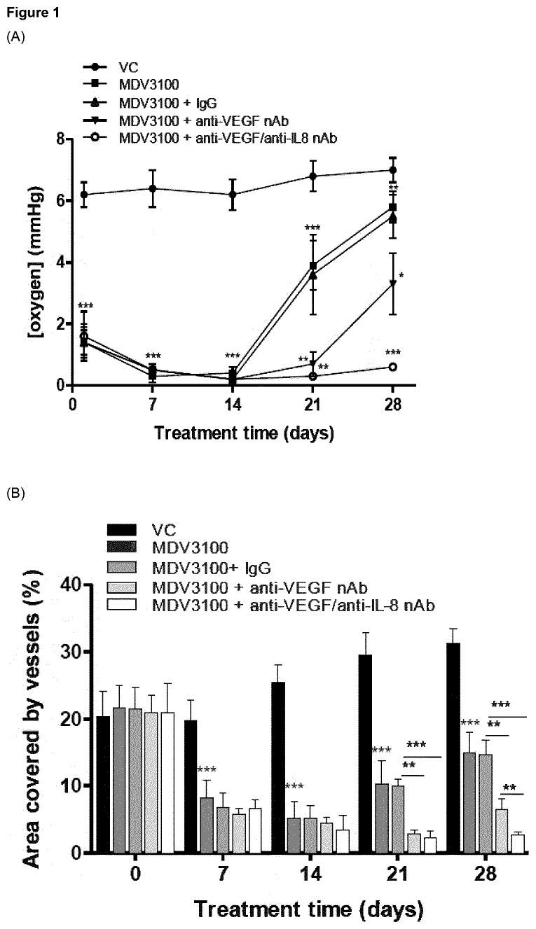

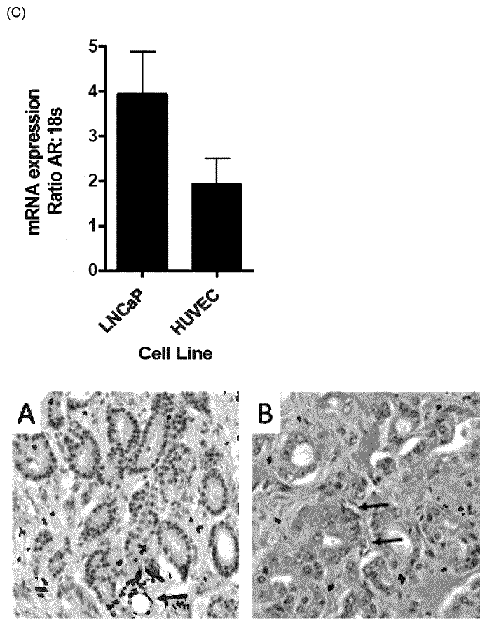

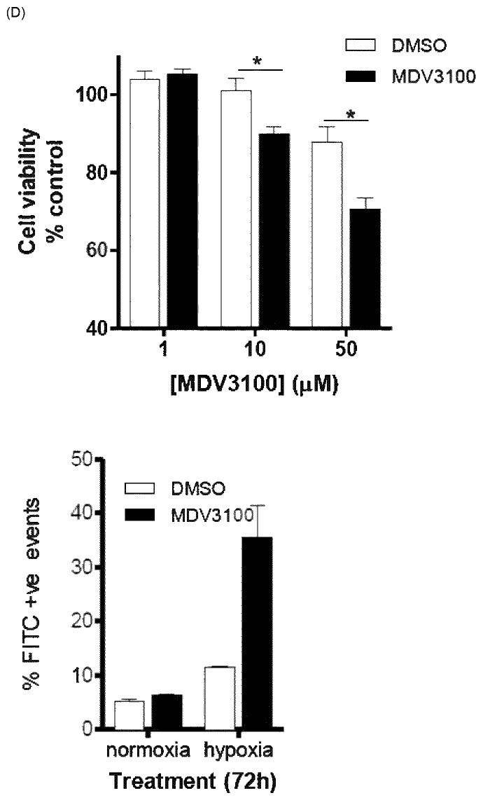

Tumour oxygenation levels were studied in an LNCaP xenograft model subjected to MDV3100 administration. Intra-tumoral oxygen levels were measured at pre-determined intervals (FIG. 1A). MDV3100 induced a rapid, profound drop in tumour oxygen levels over the initial 14 days, after which oxygen levels gradually recovered to levels observed in vehicle control (VC). Neutralizing antibodies (nAb) were administered 3 times per week to determine the role of VEGF and IL-8 signalling in elevating oxygen levels after the 14 day time-point. Anti-VEGF nAb/MDV3100 increased the duration of the hypoxic microenvironment, however, oxygen levels subsequently increased towards levels measured in VC tumours. The combination of anti-VEGF nAb and anti-IL-8 nAb with MDV3100 resulted in tumour oxygen levels remaining low for 28 days on treatment.

Using a dorsal skin flap (DSF) model, we assessed the percentage area covered by tumour vessels in all treatment groups (FIG. 1B). Vessel coverage was approximately 20% in all treatment groups at treatment initiation. Vessel density increased with time in vehicle control (VC) tumours, reaching an average value of 31±2% at day 28. In contrast, tumours treated with MDV3100 exhibited a significant reduction in vessel coverage after 7 days of treatment (8.2±2.3%; p=0.001 vs VC) which persisted out to 14 days (5.2±2.2%; p<0.0001 vs VC). An angiogenic burst was observed in MDV3100-treated animals between 14-21 days, wherein vessel coverage increased to 10.3±3.1% by day 21 and 14.7±2.6% by day 28. Vessel coverage remained suppressed to 6.6±1.3% at day 28 following administration of anti-VEGF nAb/MDV3100 (p<0.0001 vs IgG/MDV3100). The combination of anti-VEGF nAb/anti-IL-8 nAb/MDV3100 was most effective in suppressing vessel density and inhibiting tumour revascularization; vessel coverage at day 28 was 2.7±0.3% (p=0.009 vs anti-VEGF nAb/MDV3100).

To further understand the events contributing to MDV3100-promoted hypoxia, we studied the direct effect of MDV3100 administration upon vascular endothelial cells using two independent i assays. In agreement with previous studies, we detected AR mRNA in HUVEC endothelial cells by qPCR analysis (FIG. 1C, top panel). Moreover, immunohistochemical analysis of human prostate tissue detected expression of the AR protein in vascular endothelial cells (FIG. 1C, bottom panel). Subsequently, the sensitivity of human-derived vascular endothelial cells to MDV3100 was further evaluated. Administration of MDV3100 significantly reduced the viability of HUVEC cells by 10% (10 μM, p=0.025) or 17% (50 μM, p=0.011) following 72 h in vitro (FIG. 1D, top panel). Furthermore, Annexin V staining revealed an increase in the number of apoptotic cells following treatment of HUVEC cells with MDV3100 under hypoxic conditions. The induction of apoptosis by MDV3100 was minimal under normoxic culture conditions but was significantly enhanced under conditions of hypoxia (FIG. 1D, bottom panel).

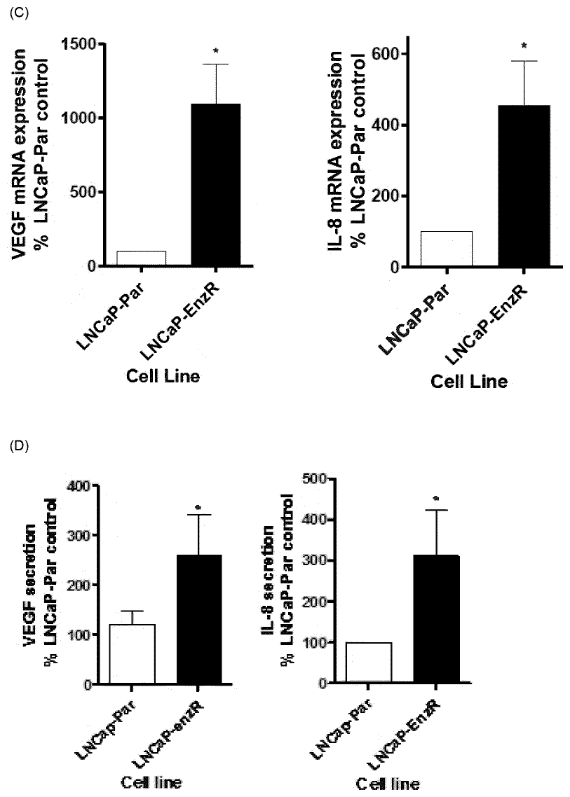

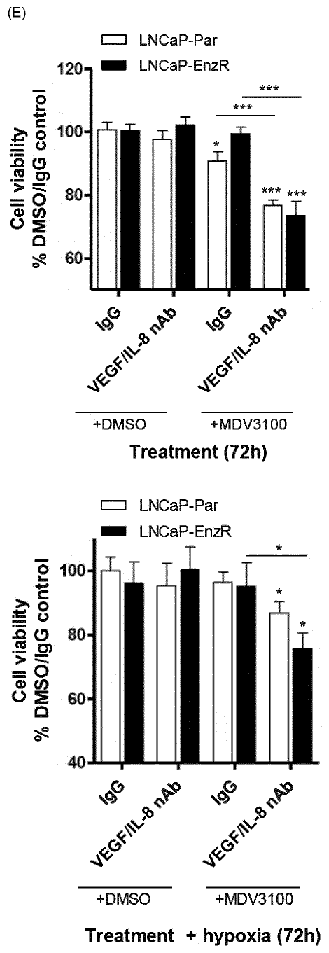

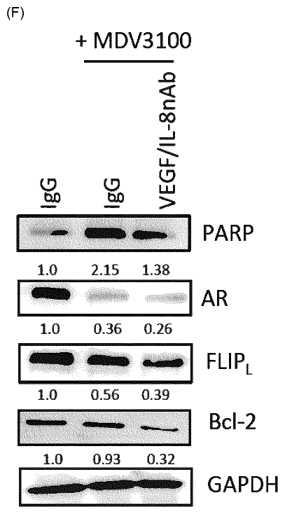

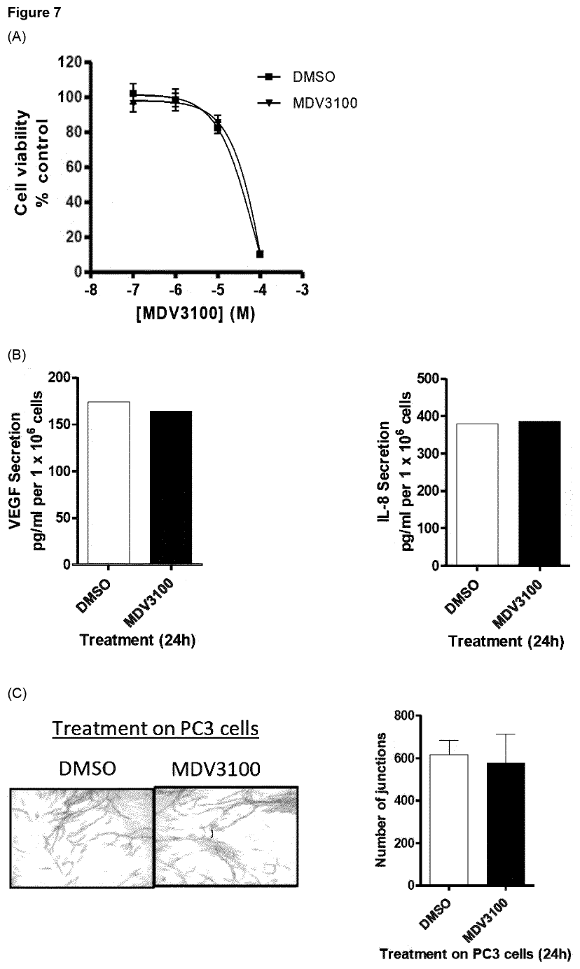

The effect of MDV3100 on blood vessel formation was examined. MDV3100 significantly impaired the development of branching junctions detected by an in vitro vascular tubule formation assay conducted over 10 days (FIG. 1E, top panel). The effect of MDV3100 on blood vessel formation was examined. MDV3100 significantly impaired the development of branching junctions detected by an in vitro vascular tubule formation assay conducted over 10 days (FIG. 1E, middle panel). To further address the effect of MDV3100 on vascular endothelial cells, we conducted in vivo assays employing the AR-null PC3 prostate cancer cells in the chick embryo experimental metastasis model, where efficiency of tissue colonization depends on the integrity of the endothelial barrier that tumour cells encounter during the extravasation phase of colonization. We confirmed that PC3 cells are unresponsive to MDV3100 relative to DMSO vehicle control in viability assays (FIG. 7A) and subsequently focused on how MDV3100 administration affected tissue colonization of the AR-negative PC3 cells. We observed that MDV3100 significantly enhanced PC3 colonization over five days in the CAM assay (p=0.011, FIG. 1E, bottom panel). Since the conditioned medium (CM) from MDV3100-treated PC3 cells had no detrimental effect on tubule formation in our in vitro angiogenesis assay (FIG. 7), it is conceivable that MDV3100-mediated enhancement of PC3 colonization in vivo involved direct MDV3100 effects on the vasculature at the sites of initial cell extravasation. Together our in vitro and in vivo results imply that it is direct rather than indirect action of MDV3100 that disrupts the vascular endothelium and underpins the enhanced colonization of PC3 cells observed in the CAM.

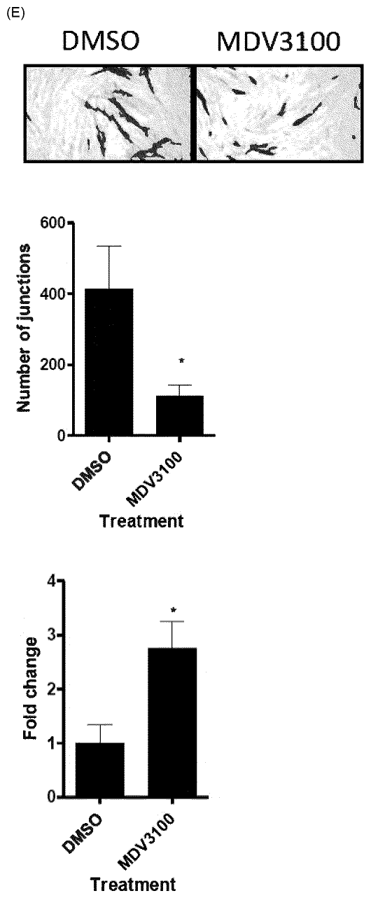

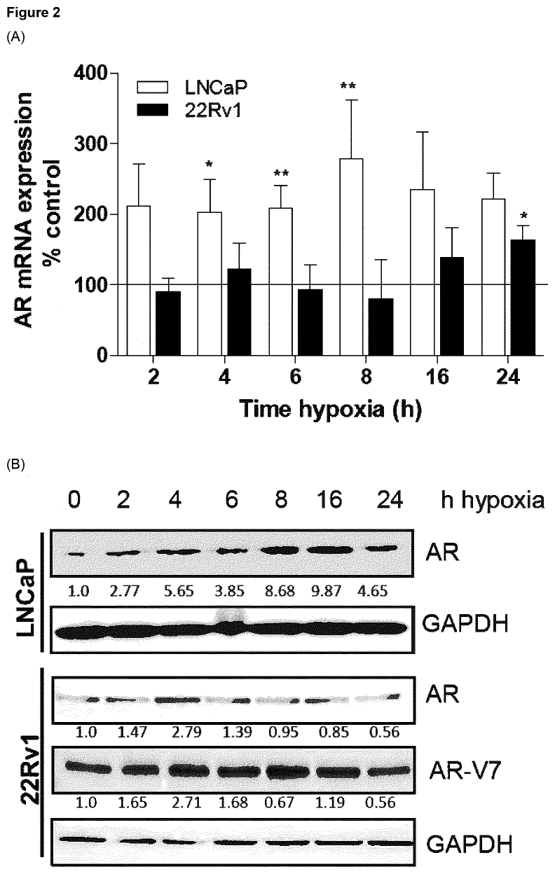

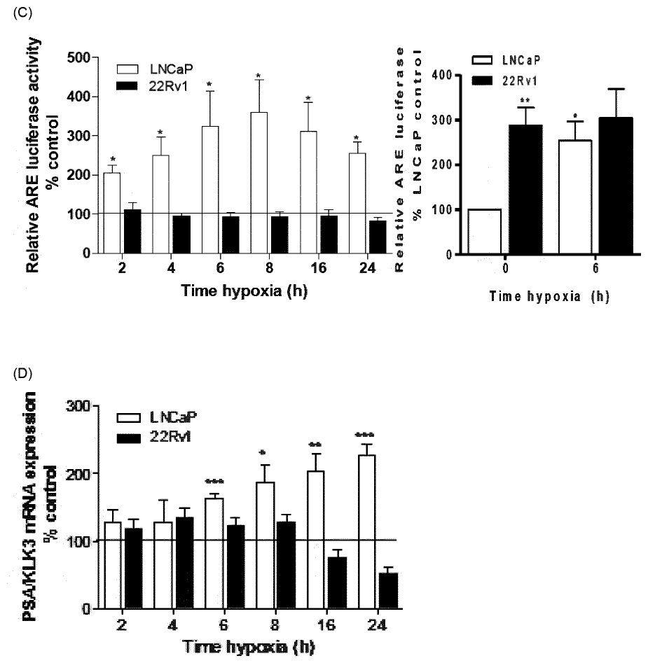

Hypoxia Potentiates and Sustains AR Expression and Activation in Prostate Cancer Cells The effect of hypoxia in modulating the AR signalling pathway was determined in vitro. Exposure of LNCaP cells to hypoxia over a 24 h time-course increased AR mRNA (FIG. 2A) and protein expression (FIG. 2B) and increased the cytoplasmic-to-nuclear translocation of the AR (FIG. 2E). Hypoxia also induced a time-dependent increase in AR activity as demonstrated by luciferase activity assays (FIG. 2C, left panel) and increased expression of the AR regulated gene PSA/KLK3, (FIG. 2D). In contrast, following an early, <3-fold increase in AR protein levels (full length and AR-V7 variant, FIG. 2B) and dampened target gene expression (FIG. 2D) in hypoxic 22Rv1 cells, levels returned to baseline. Luciferase reporter assays demonstrated that hypoxia did not potentiate AR activity in 22Rv1 cells (FIG. 2C, left panel), although parallel assays confirmed a higher basal level of AR-luciferase activity in these cells relative to LNCaP cells (FIG. 2C, right panel). Moreover, basal nuclear AR distribution was elevated in 22Rv1 cells (FIG. 2e), consistent with the higher basal levels of transcriptional activity. This data suggests that environmental hypoxia can increase intrinsic AR activity in circumstances where activity can be further potentiated by ligand-independent mechanisms.

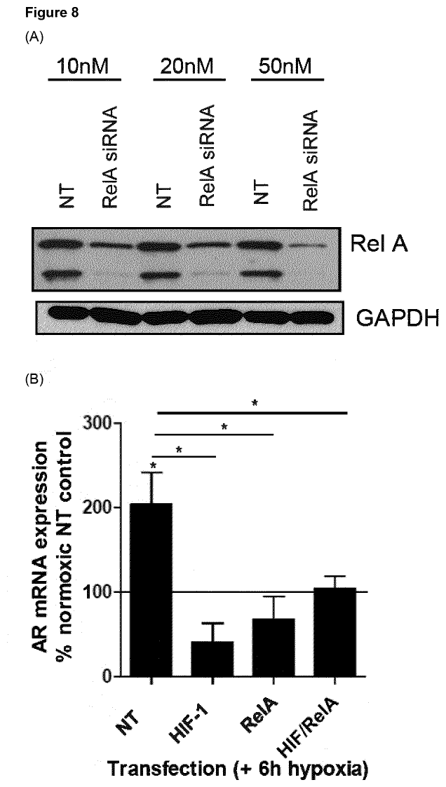

The mechanism underpinning hypoxia-induced AR expression was investigated further. Promotion of HIF-1 and NF-κB-driven transcription is a well-established response to hypoxia. Attenuation of p65RelA-driven transcription using siRNA reduced AR mRNA expression, AR protein expression and decreased PSA/KLK3 mRNA expression in LNCaP cells (FIG. 8). As previously reported, we observed that siRNA-mediated inhibition of the HIF-1α subunit was also associated with reduced AR mRNA and protein expression levels and reduced PSA/KLK3 mRNA expression in LNCaP cells. Our data suggest that treatment-induced hypoxia-promoted HIF-1 and NF-κB transcription co-operate to elevate AR expression and activity, consistent with a classical target-associated mechanism of resistance.

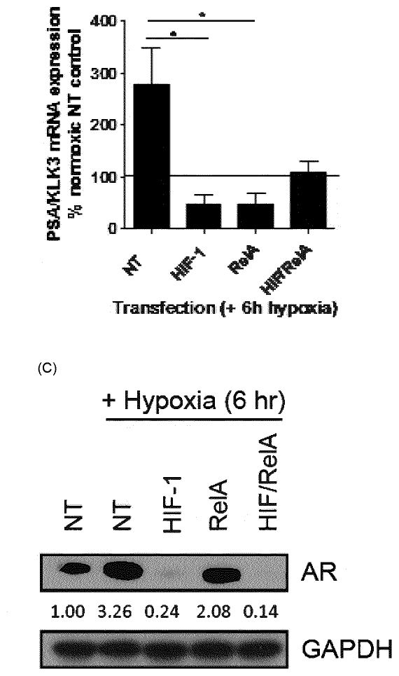

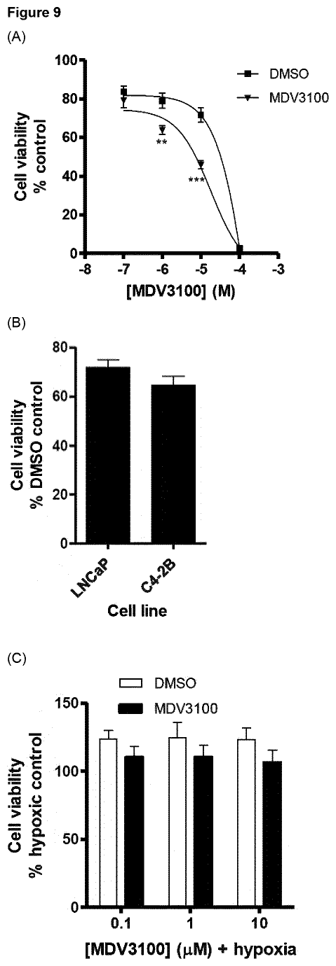

The effect of environmental hypoxia and its potentiation of AR signalling upon the pharmacology of MDV3100 was explored by initial experiments conducted on LNCaP cells cultured under normoxic or hypoxic conditions. Cells were treated with MDV3100 for 72 h prior to measurement of cell viability. While MDV3100 reduced the viability of cells cultured in normoxia relative to the DMSO control (p<0.05), the same concentration of MDV3100 was ineffective in LNCaP cells subjected to hypoxic conditions (FIG. 2F). While expression of cleaved PARP by these cells was undetectable under the experimental conditions employed, immunoblotting confirmed the loss of full-length PARP protein, indicative of PARP cleavage, in response to MDV3100 treatment of normoxic LNCaP cells but stabilization of PARP in MDV3100-treated hypoxic LNCaP cells (FIG. 2G). Similarly, the aggressive C4-2B cell line showed intermediate sensitivity to MDV3100 administration under normoxic culture conditions (FIG. 9A/B), and reduced sensitivity to MDV3100 administration under hypoxia (FIG. 9C).

### Hypoxia-Induced Signalling Sustains Hallmarks of Aggressive Cancer

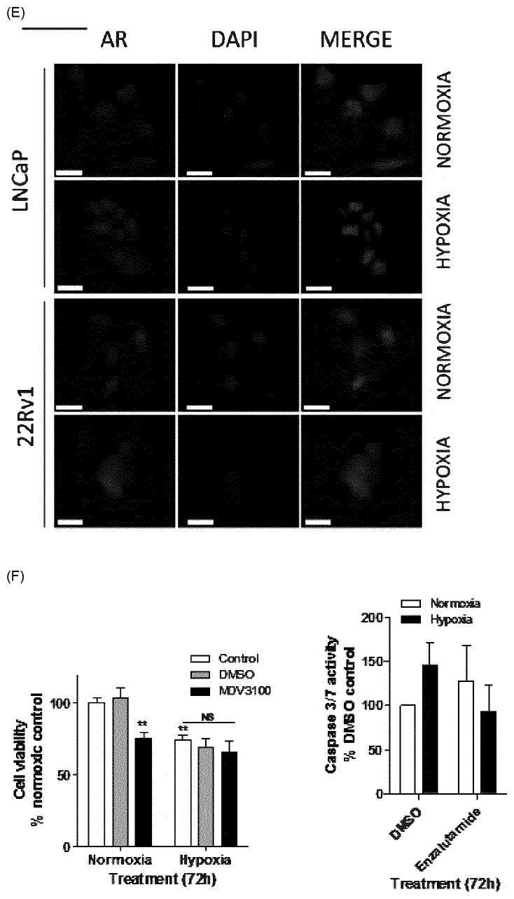

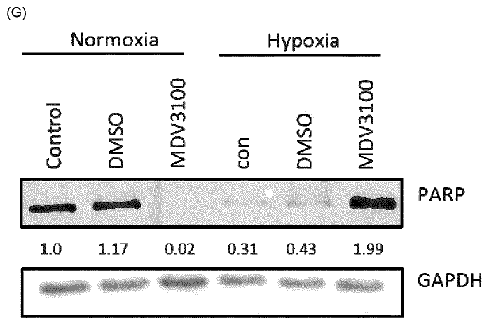

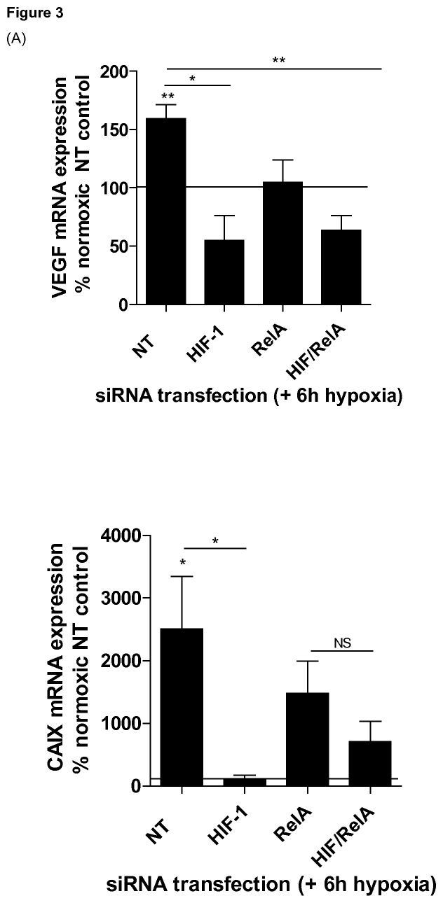

We postulated that treatment-induced hypoxia would have the potential to regulate expression of multiple genes regulating diverse biological processes including angiogenesis (VEGF), metabolic adaptation (carbonic anhydrase IX(CAIX)) and cell survival (BCL2). qPCR analysis on hypoxic LNCaP cells confirmed the induction of each of these genes and demonstrated the important contribution of HIF-1α and RelA (NF-κB) signalling to their up-regulation under hypoxia (FIG. 3A). Further experiments were conducted to investigate whether the “on-target” effects of MDV3100 (i.e. repression of AR activity) could overcome the “off-target” hypoxia-promoted HIF-1α and RelA (NF-κB) transcription effects observed in LNCaP cells. Relative to the DMSO vehicle control, MDV3100 treatment failed to attenuate hypoxia-induced expression (FIG. 3B) and secretion (FIG. 3C) of the two pro-angiogenic factors, VEGF and IL-8, by hypoxic LNCaP cells. MDV3100 also failed to reverse the hypoxia-induced up-regulation of the anti-apoptotic gene BCL2 (FIG. 3D) or that of CAIX (FIG. 3E) in LNCaP cells. This suggests that the principal pharmacological effect of MDV3100 is unable to attenuate the “off-target” hypoxia-driven increase in expression of genes associated with critical hallmarks of cancer within the microenvironment of MDV3100-treated prostatic tumours.

### Promotion of VEGF and IL-8 Signalling Contributes to Cell Survival Under Conditions of Treatment-Induced Hypoxia

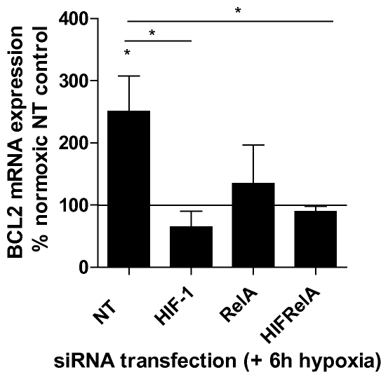

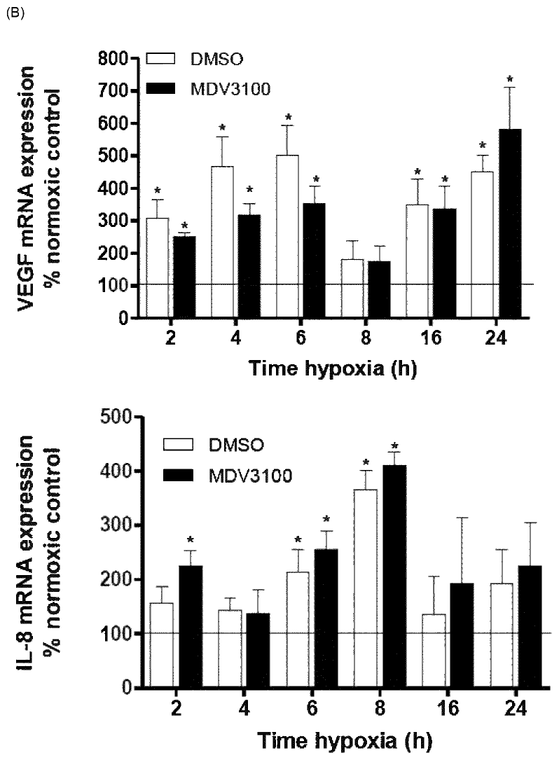

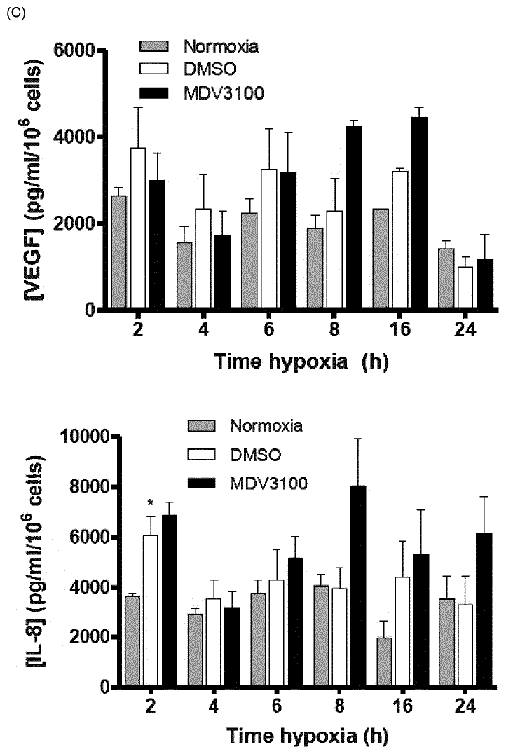

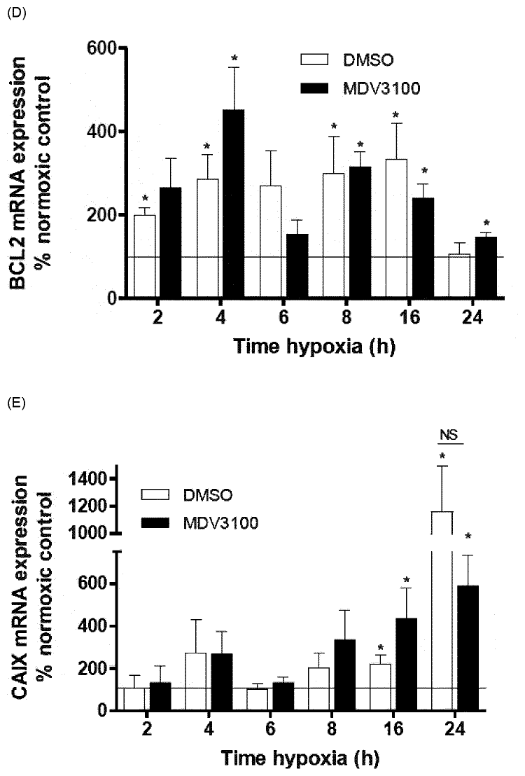

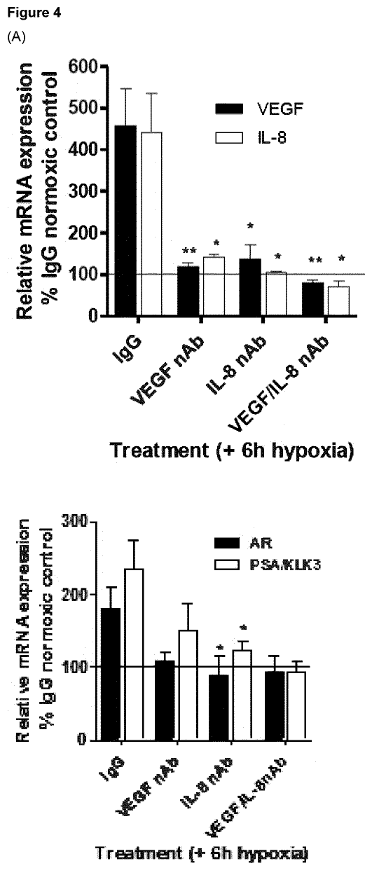

VEGF and IL-8 represent a prototypical growth factor and chemokine, respectively, inducing a plethora of signalling responses in malignant and non-malignant cells. We conducted further experiments using neutralizing antibodies against each of these factors to determine the biochemical significance of hypoxia-induced VEGF and IL-8 in sustaining hypoxia-induced gene transcription in prostate cancer cells. Administration of either the anti-IL-8 or anti-VEGF nAbs reduced hypoxia-promoted IL-8 and VEGF mRNA expression (FIG. 4A, top panel) and secretion (FIG. 4B) in LNCaP cells. Importantly, co-administration of these antibodies also repressed the expression and secretion of each of these hypoxia-induced, disease-progressing genes. Moreover, administration of the anti-IL-8 nAb partially reversed VEGF secretion, and vice-versa (FIG. 4B), demonstrating the interaction between these two angiogenic factors and the capacity to induce a feed-forward loop. With respect to the hypoxia-regulated modulation of the AR pathway, administration of anti-IL-8 or anti-VEGF nAbs attenuated the acute hypoxia-induced AR and PSA mRNA increases (FIG. 4A, bottom panel) observed in LNCaP cells. In contrast, co-inhibition of IL-8 and VEGF signalling did not modulate expression of the full-length AR or AR-V7 (FIG. 4C, top panel) and had no effect on PSA/KLK3 mRNA expression (FIG. 4C, bottom panel) detected in hypoxic 22Rv1 cells. In further experiments, we observed that the administration of anti-VEGF and/or anti-IL-8 nAbs, alone or in combination, significantly attenuated hypoxia-induced increases in Bcl-2 mRNA expression in LNCaP cells (FIG. 4D, top panel).

The importance of these signalling factors to survival under hypoxic conditions was determined by cell viability analysis. We observed that inhibition of VEGF signalling, either alone or in combination with the anti-IL-8 nAb significantly reduced the number of viable hypoxic LNCaP cells but not 22Rv1 cells (FIG. 4D, bottom panel). Together, these data support the role of these cytokines in sustaining the hypoxia-induced increase in AR pathway expression/activity and survival, establishing their relevance in promoting multiple adaptive cellular events associated with the hallmarks of castrate-resistant cells.

### Promotion of VEGF and IL-8 Signalling Contributes to Angiogenesis Under Conditions of Treatment-Induced Hypoxia

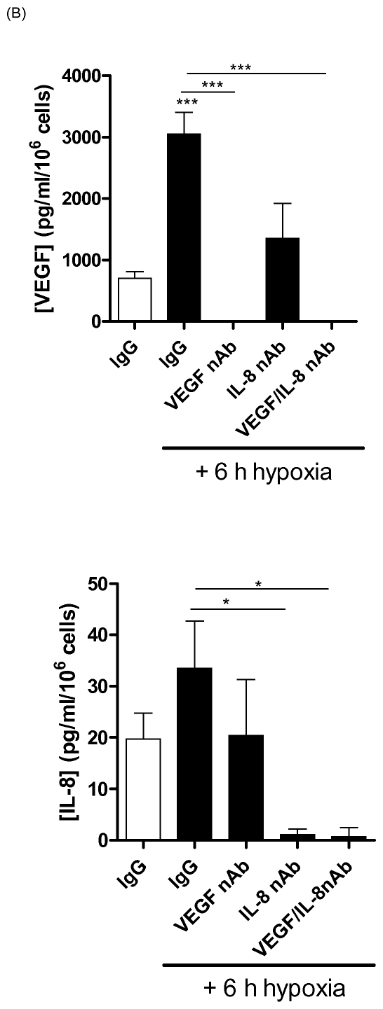

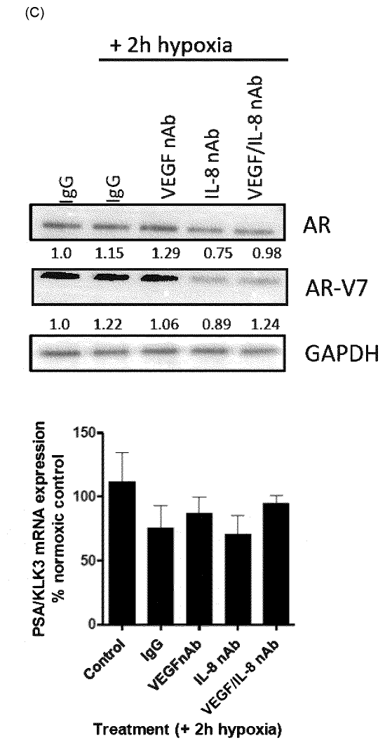

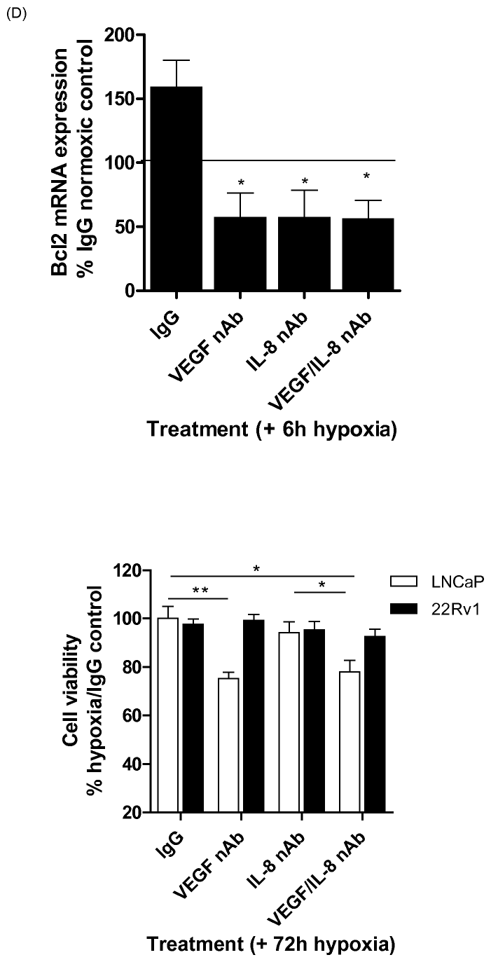

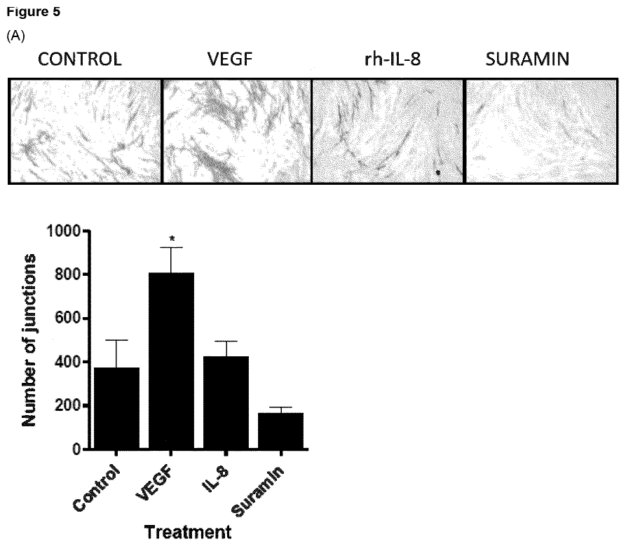

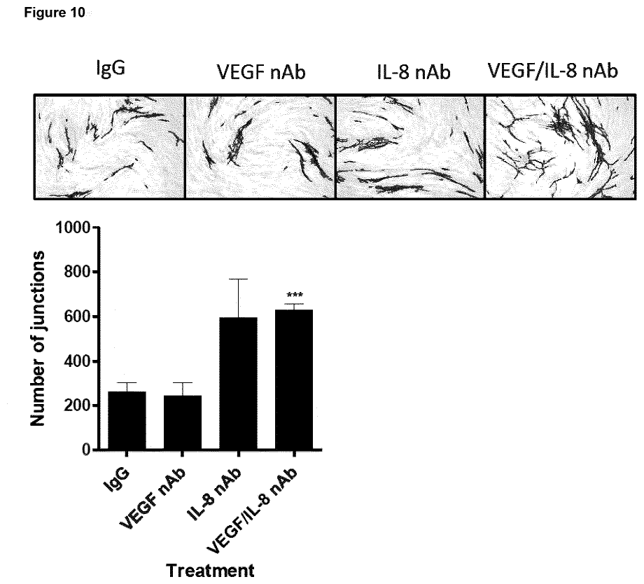

VEGF and IL-8 are known to be associated with promotion of angiogenesis. Using an in vitro tubule formation assay, we observed that administration of VEGF directly stimulated endothelial cell tubule formation while addition of exogenous rh-IL-8 was less potent in stimulating tubule formation in this assay (FIG. 5A). In a second series, the conditioned media (CM) harvested from LNCaP cells cultured in hypoxia for 24 h significantly stimulated endothelial cell tubule formation (p=0.0019, FIG. 5B). Administration of the anti-VEGF nAb, either alone (p<0.01) or in combination with anti-IL-8 nAb (p<0.01) attenuated the hypoxic LNCaP-derived CM-promoted endothelial cell tubule formation. In contrast, direct treatment of the endothelial cells with these antibodies did not attenuate the basal rate of tubule formation observed in the assay (FIG. 10), supporting the importance of the tumour cells as the source of stimulatory growth factors.

### Inhibition of VEGF and IL-8 Enhances the Response to MDV3100 In Vivo

To further characterize the importance of these specific pro-angiogenic factors in modulating the response of MDV3100-treated tumours, we examined tumour growth dynamics of an LNCaP xenograft model (FIG. 5C). In VC-treated animals, all tumours grew steadily throughout the study with a doubling time of approximately 7 days. Tumour control was observed in all MDV3100-treated groups for up to 14 days, beyond which regrowth occurred. Tumours grew rapidly in both the “MDV3100 only” and “MDV3100+IgG” control treatment groups: at treatment-day 28 (when VC animals were culled), MDV3100 (+/−IgG) treatment caused 59.5% reduction in tumour (see Table 1 below, FIG. 5C); addition of the anti-VEGF nAb to MDV3100 resulted in 76.3% reduction in tumour volume. However, the greatest delay in tumour growth was observed following combined treatment with MDV3100/anti-VEGF nAb/anti-IL-8 nAb; this combination caused 98.9% reduction in tumour growth over 28 days. Statistical analysis confirmed that combination with the anti-VEGF (p=0.0002) or the anti-VEGF/anti-IL-8 nAbs (p<0.0001) were significantly different to the MDV3100+IgG group (Table 2).

TABLE 1
Presentation of measured tumour volumes
across all treatment groups.

| | Tumour volume at day | |
|---|---|---|
| Treatment group | 28 (mm³) (±SD) | % T/C* |
| Vehicle only | 856 (±44) | — |
| MDV3100 only | 423 (±90) | 39.0 |
| MDV3100 + IgG control | 424 (±76) | 40.1 (102.8) |
| MDV3100 + anti-VEGF | 311 (±33) | 23.7 (60.6) |
| antibody | | |
| MDV3100 + anti- | 153 (±23) | 1.1 (2.9) |
| VEGF/anti-IL-8 antibodies | | |

Tumours were measured 3×/week using digital calipers. The length and width of the tumour was measured and the volume calculated using the formula volume=(length×width 2)/2. *, numbers in brackets show % T/C compared to enzalutamide alone at day 28; **, control is enzalutamide alone.

TABLE 2
Statistical analysis of LNCaP xenograft tumour growth data.

| | p value | | p value | |
|---|---|---|---|---|
| | (compared to | | (compared to | |
| | MDV3100 + | Signif- | MDV3100 + anti- | Signif- |
| Treatment | IgG control) | icance | VEGF nAb) | icance |
| MDV3100 only | >0.99 | n/s | — | — |
| MDV3100 + anti- | 0.0002 | **** | — | — |
| VEGF antibody | | | | |
| MDV3100 + anti- | <0.0001 | **** | <0.0001 | **** |
| VEGF/anti-IL-8 | | | | |
| antibody | | | | |

Analysis was performed using a 2-way ANOVA with Bonferroni post-tests, comparing the statistical significance of measured observations between treatment groups. ****, p<0.0005

Average tumour weights were also recorded at the end of the study (FIG. 5D). Relative to VC, tumour weight was significant reduced following treatment with the combination of MDV3100/anti-VEGF nAb (p=0.0144) or MDV3100/anti-VEGF nAb/anti-IL-8 nAb (p=0.0009). Administration of MDV3100/anti-VEGF nAb/anti-IL-8 nAb resulted in a significantly greater reduction in residual tumour weight compared to MDV3100/anti-VEGF nAb (p=0.011). Kaplan-Meier analysis revealed that tumour-bearing animals receiving MDV3100/anti-VEGF nAb/anti-IL-8 nAb therapy had a longer survival duration compared to all other cohorts (FIG. 5E). Importantly, 3 out of 4 animals treated with MDV3100/anti-VEGF nAb/anti-IL-8 nAb did not reach an interim analysis point of two-times treatment-starting volume by the end of the study on day 35. These results are consistent with the anti-VEGF and anti-IL-8 nAbs combining to effectively repress MDV3100-induced, hypoxia-driven angiogenesis, thereby significantly prolonging the tumour growth inhibitory effects of MDV3100.

### Expression of Hypoxia-Responsive Genes Plays a Role in Resistance to MDV3100

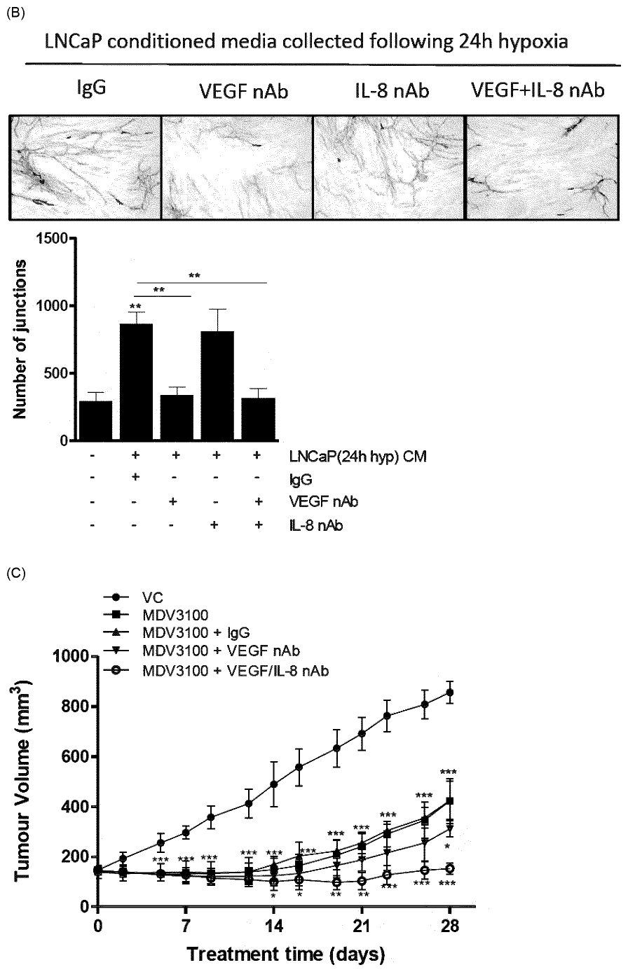

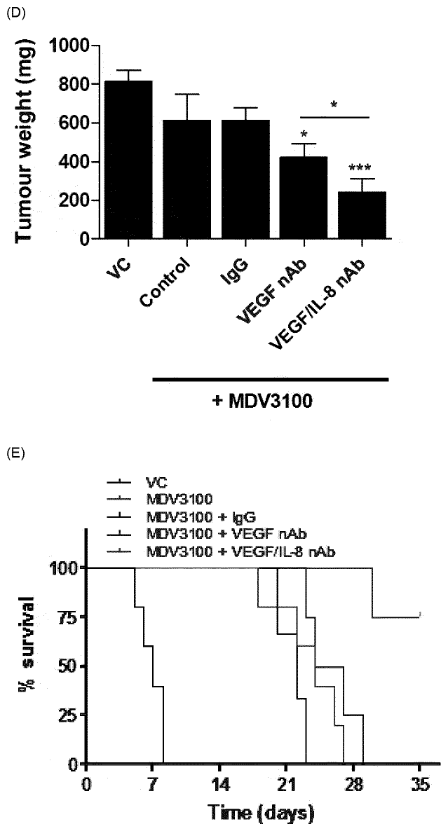

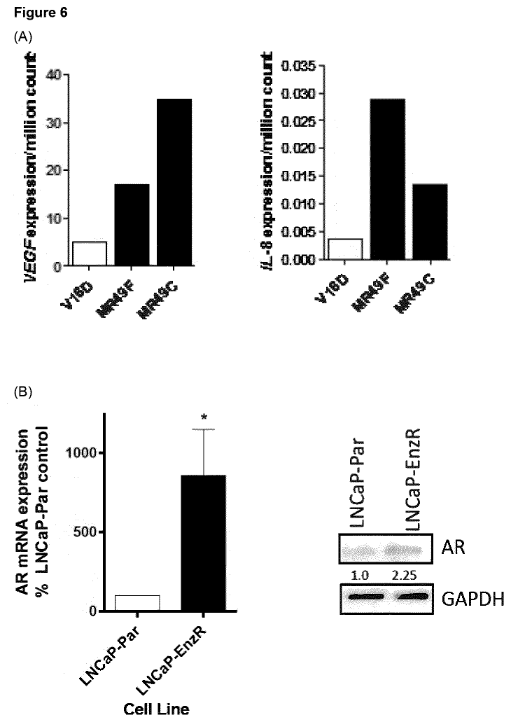

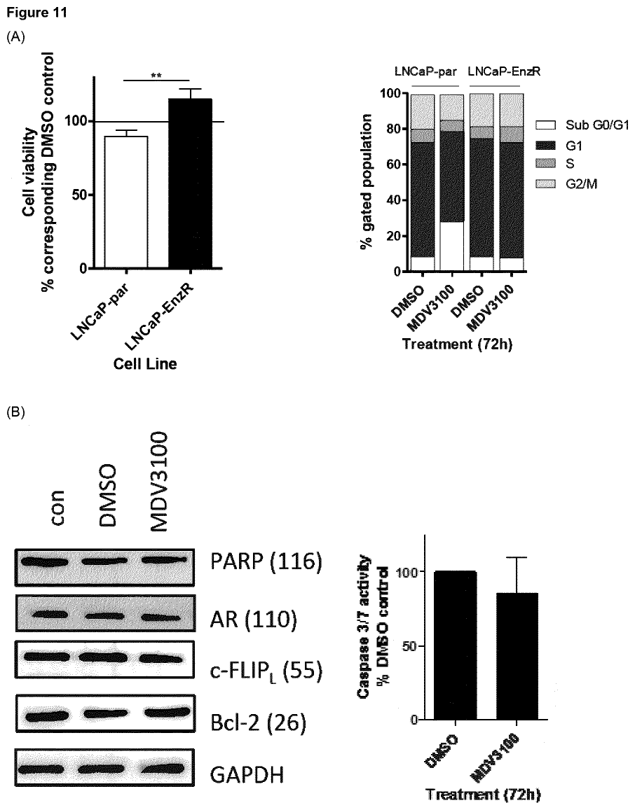

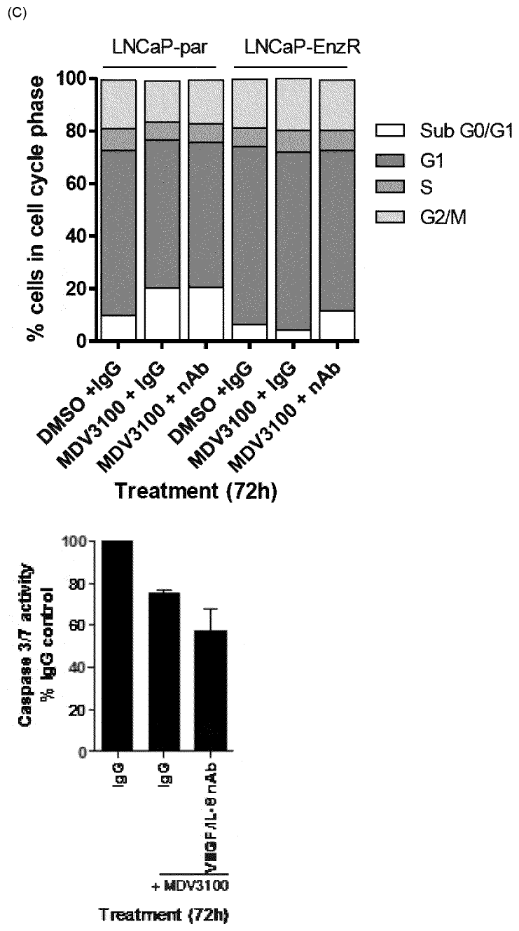

We next sought to determine whether elevated expression of IL-8 and VEGF may be detected in other experimental models of MDV3100-resistance. Elevated expression of VEGF and IL-8 mRNA expression was initially detected through analysis of a RNA-seq database characterizing gene expression in two MDV3100 resistant prostate cancer cell lines, MR49C and MR49F, originally derived through serial in vivo passaging of LNCaP xenografts treated with MDV3100 (FIG. 6A). In addition, we confirmed increased expression of the AR at mRNA and protein level (FIG. 6B), together with increased VEGF and IL-8 mRNA expression (FIG. 6C) in a further MDV3100-resistant LNCaP model, the resistant LNCaP-EnzR cells (FIG. 11), derived through continuous in vitro exposure to the AR inhibitor. ELISA also confirmed an increased secretion of IL-8 and VEGF from the LNCaP-EnzR cells relative to parental cells (FIG. 6D). The functional significance of IL-8 and VEGF signalling in modulating the resistance of these LNCaP-EnzR lines to MDV3100 was investigated using viability assays. Treatment with MDV3100 had no effect on the viability of these resistant cells relative to the DMSO control. However, co-administration with the anti-IL-8 and anti-VEGF nAbs increased the sensitivity of LNCaP-EnzR cells to MDV3100 therapy (FIG. 6E, top panel). This effect was also observed under hypoxic culture conditions (FIG. 6E, bottom panel). Immunoblotting of protein lysates confirmed that the addition of the anti-VEGF/anti-IL-8 nAbs reduced AR expression and decreased anti-apoptotic protein expression (FLIP and Bcl-2) detected in the LNCaP-EnzR line (FIG. 6F). In contrast to our prior observations in LNCaP cells (FIG. 2G), the expression of full-length PARP was clearly increased following treatment of LNCaP-EnzR cells with MDV3100 (FIG. 6F). This was partially reversed in the presence of anti-VEGF/IL-8 nAbs (FIG. 6F). Flow cytometry profiling also confirmed an increased level of apoptotic cells in those populations of LNCaP-EnzR cells subjected to MDV3100 in the presence of neutralizing IL-8 and VEGF nAbs (FIG. 11).

### DISCUSSION

The use of new generation AR antagonists including MDV3100 remains a primary treatment strategy for men with de novo systemic and advanced castrate-resistant disease. However, the development of resistance presents a major clinical problem. The present study supports the proposal that resistance to AR-targeted agents including MDV3100 may arise at least in part from development of treatment-induced hypoxia. Our in vitro and in vivo experimental data demonstrate a potent cytotoxic effect of MDV3100 on AR-expressing vascular endothelial cells and the inhibition of tubule formation, which is consistent with the observed rapid reduction in intra-tumoral oxygen levels. The ensuing hypoxia reduces the therapeutic sensitivity of prostate cancer cells to MDV3100, mediated in part through augmenting and sustaining AR expression and signalling in hypoxic conditions. Moreover, it is well established that hypoxia also induces activation of the HIF and NF-κB gene transcription pathways that underpin many hallmarks of advanced, treatment-refractory cancer. We and others have demonstrated a link between HIF-1 and/or NF-κB and AR expression and activity. Therefore we propose that the activation of these pathways provides a signalling bypass and escape mechanism to any residual on-target AR inhibition afforded by MDV3100 in hypoxic cells. For example, the hypoxia-driven, NF-κB-mediated increase in expression of the anti-apoptotic protein Bcl-2 coincides with the reduced sensitivity of hypoxic cells to MDV3100 in vitro and in vivo.

In proposing that the “off-target” hypoxia-driven changes may underpin escape from MDV3100 therapy, our further experiments focused on establishing the critical importance of two key hypoxia-regulated signalling factors in the acquisition of MDV3100 resistance. Evidence supporting the role of IL-8 and VEGF in treatment resistance is supported by our demonstration of elevated expression of these factors in LNCaP-derived MDV3100-resistant experimental models. Our data clearly show that concurrent inhibition of IL-8 and VEGF is superior in reducing MDV3100-induced expression of hypoxia-induced angiogenesis and pro-survival gene expression in vitro, in attenuating the restoration of oxygen tension in vivo, and in suppressing tumour growth and prolonging sensitivity of prostate cancer xenografts to MDV3100 therapy. Furthermore, we have shown that the concurrent repression of IL-8 and VEGF signalling can restore the sensitivity of hypoxic prostate cancer cells to MDV3100 in vitro, in part by decreasing Bcl-2 and FLIP expression and increasing MDV3100-associated apoptosis induction. Moreover, we have shown that the co-administration of anti-VEGF and anti-IL-8 nAbs restores the sensitivity of MDV3100-refractory cell lines of a LNCaP origin.

Phase III trials of anti-angiogenic compounds including Bevacizumab, Sunitinib and Lenalidomide have been discontinued due to poor efficacy in metastatic CRPC. The clinical failure of these highly-selective anti-angiogenic agents in heterogeneous disease such as CRPC may arise due to their narrow target profile and the subsequent inability to target other key factors that play a role in regulating the tumour response to hypoxia. Specifically, the proto-typical ELR+CXC-chemokine IL-8 is a potent mediator of angiogenesis, has been shown to underpin the angiogenic response of HIF-1α-deficient/VEGF-depleted DLD-1 colon cancer xenografts and mediate the resistance of head and neck squamous cell carcinoma to bevacizumab and sunitinib in renal cell carcinoma models. IL-8 expression is clinically-relevant in prostate cancer and correlates not only with microvessel density but also with shorter time-to-progression for patients being treated with hormone therapy. As demonstrated by our pre-clinical data, co-targeting of both VEGF and IL-8 signalling enhanced the MDV3100-mediated tumour control in the LNCaP xenograft model, and effectively retarded revascularization of MDV3100-treated tumours in vivo. Furthermore, co-targeting IL-8 and VEGF signalling afforded the greatest suppression of hypoxia-induced signalling and gene expression in vitro, and restored the sensitivity of hypoxic LNCaP and MDV3100 resistant LNCaP-EnzR cells to MDV3100. In totality, our data suggests that in addition to suppressing vascular responses within the tumour, the combination of anti-IL-8 and anti-VEGF nAbs also has direct impacts on adaptive MDV3100 resistance intrinsic to the tumour cell, including the repression of survival signals. It is important to note that this effective control of LNCaP tumour growth was observed using an anti-human IL-8 nAb, which restricts its action in this model to targeting only the IL-8 secreted from the implanted human tumour cells. Consequently, the actions of hypoxia-induced murine CXC-chemokine ligands (CXCL1, CXCL2 and CXCL5) originating from murine stromal and vascular endothelial cells remains intact in this experimental model and may provide some degree of functional compensation. Administration of pharmacological agents that provide total disruption of hypoxia-induced ELR+ve chemokine signalling within the prostate tumour microenvironment may yet provide even more impressive results. More importantly, our data reveal that the use of “broad-spectrum” and “multi-targeted anti-angiogenic therapies” that target both chemokines and growth factors is essential in order to derive the greatest impact in potentiating MDV3100 response in CRPC.

We conclude that treatment-induced intra-tumoral hypoxia upregulates the expression of pro-angiogenic and pro-survival factors VEGF and IL-8 which facilitate the acquisition and outgrowth of MDV3100-resistant prostate tumours. Our demonstration that combined VEGF/IL-8 inhibition significantly potentiates the prostate tumours to MDV3100 in vivo and increases sensitivity of MDV3100-resistant models provides new insights into the complex nature and multi-targeted therapeutic regimens that may be required to prolong the clinical benefit of next generation AR-antagonists in men with advanced, life-threatening prostate cancer.

The invention is not limited to the embodiments described herein but can be amended or modified without departing from the scope of the present invention as defined by the appended claim set.

1. A pharmaceutical combination for use in the treatment of cancer, the pharmaceutical combination comprising:
a vascular endothelial growth factor (VEGF) signalling inhibitor; and
an interleukin-8 (IL-8) signalling inhibitor;
wherein said use comprises administration of the pharmaceutical combination to a patient receiving androgen deprivation therapy.

2. A pharmaceutical combination for use in the treatment of cancer, the pharmaceutical combination comprising:
an androgen deprivation therapy;
a vascular endothelial growth factor (VEGF) signalling inhibitor; and
an interleukin-8 (IL-8) signalling inhibitor.

3. A pharmaceutical combination for use in potentiating a therapeutic effect of androgen deprivation therapy in the treatment of cancer, the pharmaceutical combination comprising:
a vascular endothelial growth factor (VEGF) inhibitor; and
an interleukin-8 (IL-8) inhibitor.

4. The pharmaceutical combination for use according to any one of claims 1-3, wherein the androgen deprivation therapy comprises treatment with an anti-androgen and/or an androgen signalling inhibitor.

5. The pharmaceutical combination for use according to claim 4, wherein the anti-androgen is an androgen receptor antagonist.

6. The pharmaceutical combination for use according to claim 5, wherein the androgen receptor antagonist is selected from one or more of enzalutamide (MDV3100), Apalutamide (ARN-509), Bicalutamide (Casodex), or Darulutamide.

7. The pharmaceutical combination for use according to claim 5, wherein the androgen receptor antagonist is enzalutamide (MDV3100).

8. The pharmaceutical combination for use according to claim 4, wherein the androgen signalling inhibitor is selected from abiraterone-acetate, finasteride, dutasteride, leuprolide or gooserelin.

9. The pharmaceutical combination for use according to any of the preceding claims, wherein the VEGF signalling inhibitor comprises an antibody.

10. The pharmaceutical combination for use according to claim 9, wherein the antibody comprises a VEGF neutralising antibody.

11. The pharmaceutical combination for use according to claim 9 or 10, wherein the antibody comprises bevacizumab.

12. The pharmaceutical combination for use according to any claims 1-9, wherein the VEGF signalling inhibitor comprises an inhibitor or antagonist of the VEGF receptor, optionally wherein the VEGF receptor is selected from VEGFR1 and VEGFR2.

13. The pharmaceutical combination for use according to any of the preceding claims, wherein the IL-8 signalling inhibitor comprises an antibody.

14. The pharmaceutical combination for use according to claim 13, wherein the antibody comprises an IL-8 neutralising antibody.

15. The pharmaceutical combination for use according to any claims 1-13, wherein the IL-8 signalling inhibitor comprises an inhibitor or antagonist of the IL-8 receptor, optionally wherein the IL-8 receptor is selected from CXCR1 and CXCR2.

16. The pharmaceutical combination for use according to claim 15, wherein the inhibitor or antagonist of the IL-8 receptor comprises a selective and/or non-selective small molecule or peptide/peptidomimetic inhibitor of CXCR1 and/or CXCR2.

17. The pharmaceutical combination for use according to any of the preceding claims, wherein the cancer is a cancer characterised by one or more areas of hypoxia within the cancer.

18. The pharmaceutical combination for use according to any of the preceding claims, wherein the cancer is a cancer characterised by increased expression of VEGF and/or IL-8.

19. The pharmaceutical combination for use according to any of the preceding claims, wherein the cancer is selected from prostate cancer or breast cancer.

20. The pharmaceutical combination for use according to any of the preceding claims, wherein the cancer is prostate cancer.

21. The pharmaceutical combination for use according to claim 20, wherein the pharmaceutical combination is administered to prostate cancer within primary site of the cancer or to an extra-prostatic site.

22. The pharmaceutical combination for use according to any one of the preceding claims, wherein the prostate cancer is hormone-naïve, hormone-sensitive or castrate-resistant prostate cancer.

23. The pharmaceutical combination for use according to any one of the preceding claims, the cancer is refractory to treatment with an anti-cancer therapy.

24. The pharmaceutical combination for use according to claim 23, wherein the anticancer therapy is selected from one or more of a chemotherapeutic agent, radiotherapy, and androgen deprivation therapy.

25. The pharmaceutical combination for use according to claim 24, wherein the androgen deprivation therapy comprises treatment with an anti-androgen and/or an androgen signalling inhibitor.

26. The pharmaceutical combination for use according to claim 25, wherein the anti-androgen is an androgen receptor antagonist.

27. The pharmaceutical combination for use according to claim 23, wherein the androgen receptor antagonist is selected from one or more of enzalutamide (MDV3100), Apalutamide (ARN-509), Bicalutamide (Casodex), or Darulutamide.

28. The pharmaceutical combination for use according to claim 26, wherein the androgen receptor antagonist is enzalutamide (MDV3100).

29. The pharmaceutical combination for use according to claim 25, wherein the androgen signalling inhibitor is selected from abiraterone-acetate, finasteride, dutasteride, leuprolide or gooserelin.

30. The pharmaceutical combination for use according to any of the preceding claims, wherein radiotherapy is used in combination with the androgen deprivation therapy.

31. The pharmaceutical combination for use according to claim 24 or 30, wherein the radiotherapy is selected from one or more of external beam radiation therapy, brachytherapy (sealed source radiotherapy), unsealed source radiotherapy (systemic radioisotope therapy), intraoperative radiotherapy, deep inspiration breath-hold radiotherapy or radionuclide therapy (e.g. radium-223).
